(19) United States
(12) Patent Application Publication
(10) Pub. No.: US 2025/0213695 A1
(43) Pub. Date: Jul. 3, 2025

(54) ULTRASOUND-MEDIATED GENE THERAPY FOR DEEP VEIN THROMBOSIS AND POST-THROMBOTIC SYNDROME

(71) Applicant: Cedars-Sinai Medical Center, Los Angeles, CA (US)

(72) Inventors: Dan Gazit, Los Angeles, CA (US); Eric Ley, Playa del Ray, CA (US); Zulma Gazit, Los Angeles, CA (US); Gadi Pelled, Los Angeles, CA (US)

(73) Assignee: Cedars-Sinai Medical Center, Los Angeles, CA (US)

(21) Appl. No.: 18/852,712

(22) PCT Filed: Apr. 14, 2023
(86) PCT No.: PCT/US2023/065788

§ 371 Date: Sep. 30, 2024

Related U.S. Application Data
(60) Provisional application No. 63/330,870, filed on Apr. 14, 2022.

(51) Int. Cl. A61K 41/00; A61B 8/00; A61B 8/08; A61K 38/48; A61K 48/00; A61M 37/00; A61P 7/02; C12N 9/72; C12N 15/85

(52) U.S. Cl. CPC A61K 41/0047; A61B 8/0891; A61B 8/481; A61K 38/482; A61K 38/484; A61K 48/0033; A61K 48/0058; A61K 48/0075; A61M 37/0092; A61P 7/02; C12N 9/6459; C12N 15/85; C12Y 304/21068; A61M 2205/3375; C12N 2830/15

(57) ABSTRACT

Systems and methods are provided for ultrasound-based microbubble-assisted or nanobubble-assisted theragnosis, treating, ameliorating, or preventing one or more vascular diseases. In particular, systems and methods are provided for transcutaneous imaging of blood vessels located at various depths (including deeper regions) from the skin surface using ultrasound, and/or performing localized gene therapy using ultrasound at or near the imaged blood vessels. The systems and methods described herein may be used for diagnosis and/or treatment of vascular disorders, including but not limited to DVT, PE, venous thromboembolism (VTE) that includes DVT and PE, where PE usually follows DVT, post-thrombotic syndrome, embolic strokes, embolic heart attacks, and combinations thereof.

### CROSS-REFERENCE TO RELATED APPLICATIONS

This application claims priority to and the benefit of U.S. Provisional Patent Application No. 63/330,870, filed Apr. 14, 2022, which is hereby incorporated by reference herein in its entirety.

### TECHNICAL FIELD

The present invention is directed to systems and methods for ultrasound-mediated imaging and therapy, and more particularly to imaging and treatment of vascular conditions, such as deep vein thrombosis and post-thrombotic syndrome.

### BACKGROUND

The following description includes information that may be useful in understanding the present invention. It is not an admission that any of the information provided herein is prior art or relevant to the presently claimed invention, or that any publication specifically or implicitly referenced is prior art.

Deep vein thrombosis (DVT) is the most common form of venous thrombosis and can lead to pulmonary embolism (PE). It is estimated that DVT and PE affect 300,000-600,000 people annually in the United States, resulting in the death of 60,000 to 100,000 of these patients. Recently, the risk of DVT and PE has been shown to be doubled in critically ill COVID-19 patients. In the long term, the main risk for proximal thrombosis is the appearance of post-thrombotic syndrome (PTS) in about 20-50% of cases, characterized by functional impotence, pain, pruritus and/or distal trophic disorders, with a major alteration of quality of life. Current standard of care includes: (i) anticoagulation treatments, which are often inefficient for recanalizing the occluded vessel; (ii) thrombolysis, mainly using tissue plasminogen activator (tPA), which is associated with a 10% rate of severe hemorrhagic events, and requires prolonged time for effect, making the treatment physically taxing on patients and increasing medical costs; (iii) endovascular invasive recanalization, which is associated with substantial risk of hematoma, false aneurysm, re-occlusion, stent rupture, clot detachment and infection; and iv) thrombectomy, which is invasive and increases the risk of bleeding and PE.

Some approaches employ ultrasound-based thrombolysis; however, they are limited by one or more of potential damage to blood-vessel walls, low efficiency of clot removal, invasiveness (attributed to catheter-based devices), and low efficiency of tPA delivery. Hence, there is a clear unmet need for the development of novel targeted therapies to safely and effectively treat DVT in a non-invasive or minimally invasive manner.

SUMMARY

Systems and methods are provided for ultrasound-based treatment of vascular diseases conditions including but not limited to DVT, PE, venous thromboembolism (VTE) that includes DVT and PE, where PE usually follows DVT, post-thrombotic syndrome, embolic strokes, embolic heart attacks, and combinations thereof. The Centers for Disease Control and Prevention (CDC) indicates that up to 900,000 Americans could be affected by DVT per year. Further, sudden death is the first symptom in about 25% of patients who develop PE. Furthermore, DVT may develop from a number of causes. The strongest risk factors include trauma or fracture, and major orthopedic or oncological surgeries. Moderate to weak risk factors include hypercoagulopathy, other surgeries, and prolonged immobility. Approximately 5-8% of the U.S. population has one of several genetic risk factors, known as inherited thrombophilias, which increases the risk for thrombosis. It is therefore crucial to diagnose and treat DVT rapidly to minimize potential complications.

Previous approaches for treatment of DVT include anticoagulant medications, which aim to reduce the risk of thrombi and PE, while allowing physiological thrombolysis to occur. Some other approaches employ thrombolytic agents such as recombinant tissue plasminogen activator (rtPA) or urokinase, which can be given systemically or directly to the thrombus site via a catheter. The thrombus can also be removed surgically (thrombectomy) or via endovascular mechanical techniques. Further, some approaches combine pharmaceutical and mechanical methods to better introduce the thrombolytic agent to the thrombus. However, the inventors herein have recognized that these treatments have significant drawbacks, which are summarized in Table 1 below. In general, anticoagulants and thrombotic factors increase the risk of bleeding, while techniques that include mechanical methods are invasive, time consuming, and could lead to emboli and venous damage.

TABLE 1
Current treatments for DVT

| Treatment | Drawbacks and adverse events |
|---|---|
| Systemic anticoagulants | Increased risk of bleeding (35); long |
| | treatment time (at least three months) (36); |
| | does not dissolve thrombus, does not prevent |
| | venous damage or subsequent post- |
| | thrombotic syndrome (37) |
| Systemic thrombolytic therapy | Increased risk of bleeding; requires ICU |
| | admission, long hospitalization, numerous |
| | laboratory tests (38) |
| Surgical thrombectomy | Used in limited number of patients; difficult to |
| | achieve venous patency and preservation of |
| | venous valves (38) |
| Catheter-directed thrombolysis | Intracranial bleeding (although rare) (39); long |
| | infusion time, with intensive monitoring and |
| | long hospitalization (38) |
| Pharmacomechanical catheter-directed | Risk of damage to venous valves |
| thrombolysis | |
| Percutaneous mechanical thrombectomy | Increase risk of PE |
| Percutaneous aspiration thrombectomy | No standardized protocol, not widely accepted |
| Mechanical filters to prevent emboli | Risk of preformation and retroperitoneal |
| | bleeding, filter embolization, and filter fracture |
| | (40) |

As indicated above, an example approach to treat thrombi includes ultrasound (US) based thrombolysis, also referred to as sonothrombolysis. In this approach, US is used to induce clot dissolution when placed externally (transcutaneous) or when applied via catheter. However, the transcutaneous approach requires high precision in order to reduce damage to adjacent tissues, while the endovascular, catheter-based US transducers are invasive.

In another example approach, an endovascular device for treating PE is used to deliver microbubbles (MBs) and rtPA to enhance thrombolysis. However, randomized clinical trials showed that the endovascular device's effect was not significant, compared to localized rtPA delivery alone, post-treatment and in one-year follow-up. In addition, in the use of US in conjunction with a thrombolytic agent, treatment time still remains long (e.g., 15-45 hours) in order to achieve substantial thrombus lysis (>50%).

Yet another example approach utilizes a forward-viewing US transducer with higher acoustic pressure output and have demonstrated their efficacy in several in vitro studies. However, the above-mentioned intravascular US transducers have a relatively short focal distance (<1-1.5 mm) due to their small aperture size and relatively low operation frequency (<0.7 MHz), which could reduce cavitation effects of MBs and limit thrombolysis efficiency (<30-50% mass reduction for 30 min in vitro). In another example approach, Kim et al developed a forward-viewing, endovascular, multi-pillar piezoelectric stack transducer capable of transmitting higher acoustic pressure over a distance greater than 2 wavelengths. Therein, MBs were replaced with nanodroplets. However, Kim's approach was performed using an in vitro clot model, and may suffer from potential damaging effects to blood vessels.

Some other approaches use gene therapy to potentially provide local secretion of thrombolytic proteins over time without the need for long-term infusions in intensive care units. However, the studies did not include functional outcome measures or explore the transfected target cells post-treatment. Furthermore, the above-mentioned approaches were not performed on clinically relevant large-animal models and are not directly applicable to large-animal models.

The inventors herein have identified the above-mentioned disadvantages. Further, the inventors have recognized using ultrasound-based gene therapy may be provided to treat vascular conditions such as thrombosis, embolus, etc., effectively and safely. Furthermore, the inventors have recognized that ultrasound-based gene therapy may be used to treat vascular conditions in deep veins (e.g., deep vein thrombosis) in a non-invasive or minimally invasive manner. Further still, the inventors have identified that ultrasound contrast agent-enhanced US-based imaging of vascular disease (e.g., a thrombosis and/or embolus) in combination with ultrasound contrast agent-enhanced US-based gene delivery (e.g., for treating the thrombosis and/or embolus) may be used to image and diagnose the vascular disease as well as provide gene therapy to induce local expression of a thrombolytic factor in a blood vessel, such as a thrombosed vein, which may lead to effective thrombolysis in a non-invasive or minimally invasive manner within a reduced period of time. Accordingly, the inventors provide systems and methods for simultaneous imaging, diagnosis, and/or treatment of vascular diseases using contrast-agent- enhanced ultrasound.

### SUMMARY

As one example, provided herein is a method for theragnosis, treating, ameliorating, or preventing/reducing the likelihood of a vascular condition in a subject, the method comprising: administering a composition to the subject at or near a region of interest including one or more blood vessels, the composition comprising an ultrasound contrast agent and plasmid DNA including at least a gene for a therapeutic agent and a sequence that causes the therapeutic agent to be expressed in the subject; and imaging, via an ultrasound probe, the contrast agent in the region of interest using a contrast pulse sequence of ultrasound plane waves; and applying, via the ultrasound probe, a therapeutic ultrasound emission for transfection of the plasmid DNA into a plurality of target cells of the one or more blood vessels.

In one example of the method, the ultrasound probe is configured to image the one or more blood vessels, one or more occlusions associated with the one or more blood vessels, and/or microbubbles in the one or more blood vessels and further configured to provide transcutaneous ultrasound therapy to the one or more blood vessels to treat the vascular condition at a depth below the skin surface. In some examples, the one or more blood vessels are deep veins. In some examples, the depth may be in a range between 1 cm and 20 cm below the skin surface. In some examples, the depth may be greater than 20 cm. In some examples, the depth may between 5 cm and 10 cm. In some examples, the depth may be between 10 cm and 15 cm.

In another example, a composition for treatment of one or more vascular conditions in a subject comprises: a microbubble; and a vector including a gene encoding a therapeutic agent for the treatment of the one or more vascular conditions.

In another example, a method for theragnosis, treating, ameliorating, or preventing/reducing the likelihood of one or more vascular conditions in a subject comprises: providing a composition at or near a region of interest for the subject, the composition comprising an ultrasound contrast agent and a vector including a gene encoding a therapeutic agent for the treatment of the one or more vascular conditions; performing contrast imaging of the region of interest, via an ultrasound probe, using an imaging ultrasound sequence; and transmitting an ultrasound sequence, via the ultrasound probe, for transfection of the vector in to a plurality of target cells at or near the region of interest; wherein the ultrasound contrast agent comprises a plurality of microbubbles or a plurality of nanobubbles.

In this way, ultrasound-based gene therapy may be used for delivering targeted and/or localized treatment to vascular conditions. As a result, safe, effective, and faster treatment of vascular conditions may be achieved in a minimally invasive or non-invasive manner.

In another embodiment a kit comprises an ultrasound contrast agent comprising microbubbles or nanobubbles; a vector comprising a nucleic acid sequence encoding a therapeutic agent for treating, ameliorating, or preventing/reducing the likelihood a vascular disease; and instructions for use. In one example, the vector is a plasmid.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements and are not drawn to scale.

FIG. 1 is a block diagram of an ultrasound system, according to an embodiment of the disclosure;

FIG. 2 is a schematic illustration of an example method for theragnosis, transcutaneously treating or ameliorating one or more vascular conditions using an ultrasound probe, such as the ultrasound probe of the ultrasound system of FIG. 1, according to an embodiment of the disclosure;

FIG. 3 is a flow chart illustrating an example method for theragnosis of a vascular condition according to an embodiment of the disclosure;

FIG. 4 is a flow chart illustrating an example method for imaging and treatment by an ultrasound probe configured for theragnosis, such as the ultrasound probe of the ultrasound system of FIG. 1, according to an embodiment of the disclosure;

FIGS. 5A-5F show example Ex vivo, US-based transfection of porcine femoral vein. Femoral vein was washed with sterile PBS, and 2 cm segment ligated using sutures (A; white arrows). Catheter was inserted into vein to enable DNA and MBs delivery (B; white arrow). Following GFP plasmid and MBs injection, US was applied transcutaneously in contrast mode (C). US was applied until MBs were not visible (D). Vein segment was cultured for 48 hours and inspected using fluorescence imaging system, showing areas of strong signal from internal and external aspects of vein (E&F).

FIGS. 6A and 6B show example Overexpression of human tPA in porcine BM-MSCs. Porcine MSCs were nucleofected with 10 μg of tPA plasmid. 24- and 48-hours post-transfection quantitative RT-PCR showed high expression of transgene compared to mock-transfected cells (A, RQ=relative quantification). TPA activation assay was used to demonstrate presence of active enzyme in media of transfected cells, compared to non-and mock-transfected cell cultures (B). Results show biological replicates and technical triplicates of samples.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.” As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4.5%, 4%, 3.5%, 3%, 2.5%, 2%, 1.5%, 1%, or 0.5% of that referenced numeric indication, if specifically provided for in the claims.

As used herein, a “subject” refers to a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

As used herein, the term “real-time” is defined to include a process occurring without intentional delay. For purposes of this disclosure, the term “real-time” will additionally be defined to include an action occurring within 10 seconds. For example, if US data is acquired, a real-time response (e.g., 2D image or 3D rendering) based on that data would occur within 10 seconds of the acquisition. Those skilled in the art will appreciate that most real-time processes will be performed in substantially less time than 10 seconds.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., plasmid DNA, genomic DNA or cDNA. Suitable RNA can include, e.g., mRNA.

As used herein, the term “near real-time” is defined to include a process occurring without intentional delay, given the processing limitations of the system and the time required to acquire the data.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

The present description relates to systems and methods for ultrasound-based theragnosis, treating, ameliorating, or preventing/reducing the likelihood of vascular diseases or disorders. Theragnosis comprises diagnostic imaging and therapy, including optionally imaging at any time and for any duration from the start of initiating the ultrasound-based therapy until the ultrasound-based therapy is completed. In particular, systems and methods are provided for transcutaneous imaging of blood vessels located at various depths (including deeper regions) from the skin surface using ultrasound, and/or performing localized gene therapy using ultrasound at or near the imaged blood vessels. The systems and methods described herein may be used for diagnosis and/or treatment of vascular disorders, including but not limited to DVT, PE, venous thromboembolism (VTE) that includes DVT and PE, where PE usually follows DVT, post-thrombotic syndrome, embolic strokes, embolic heart attacks, and combinations thereof.

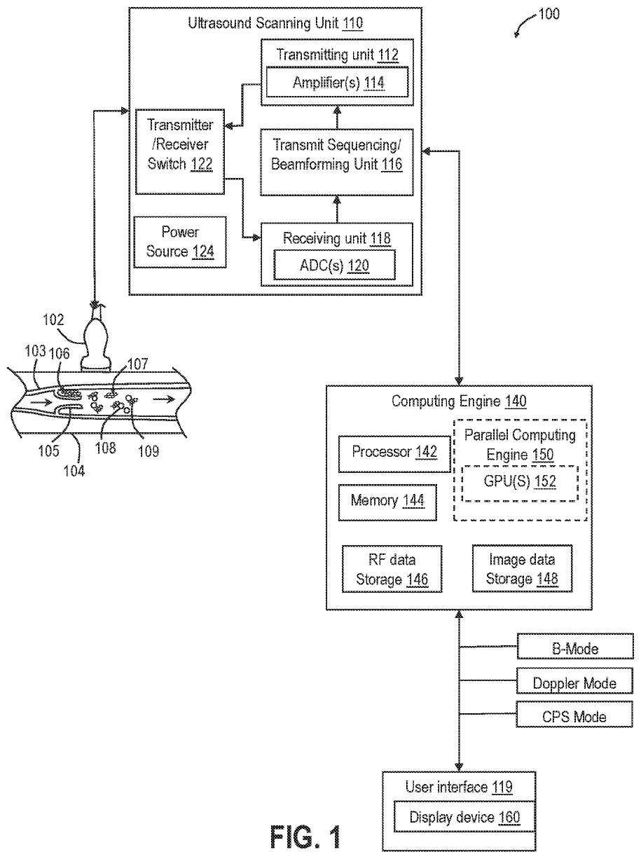

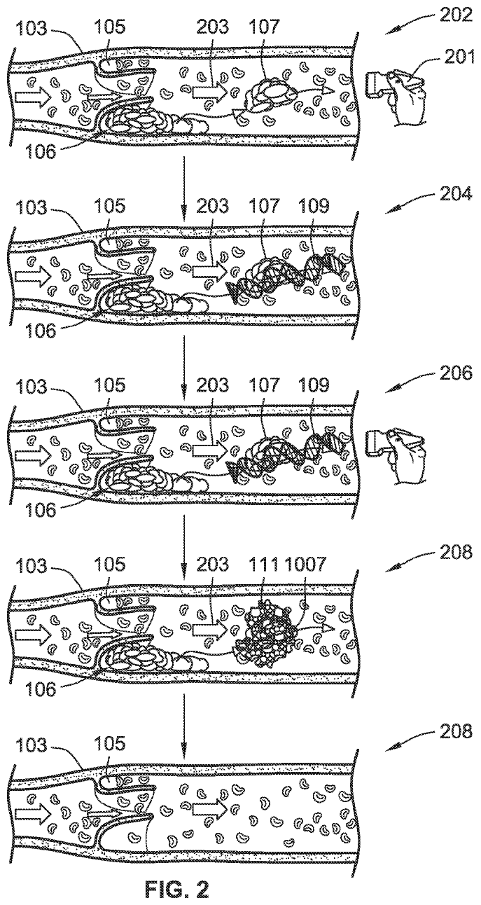

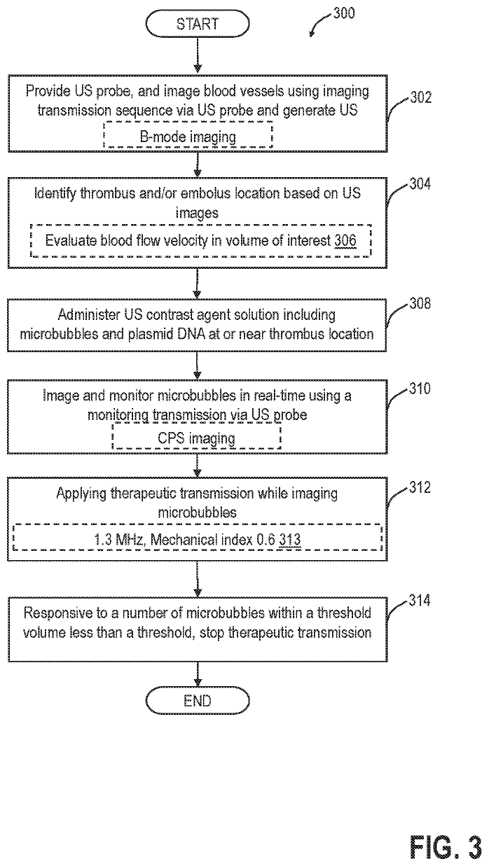

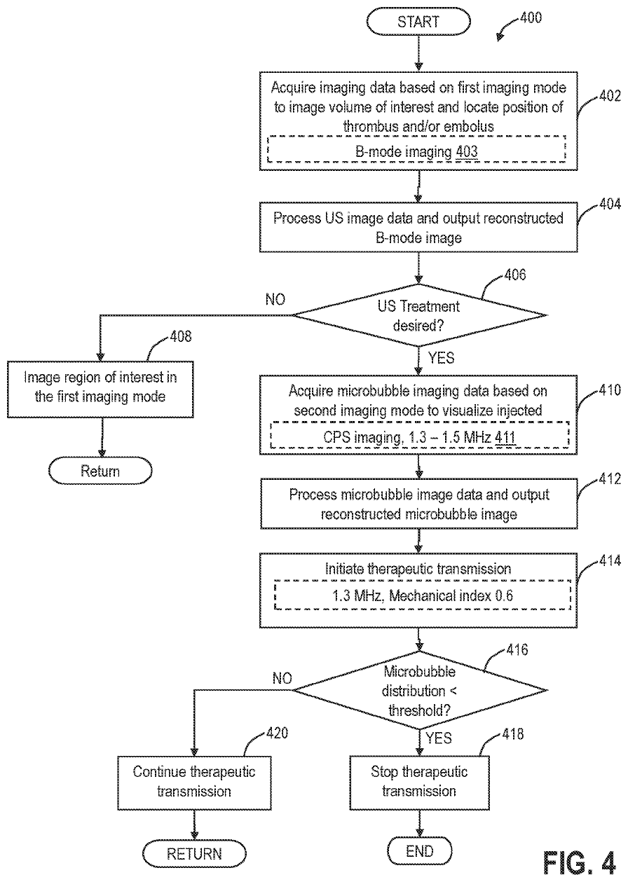

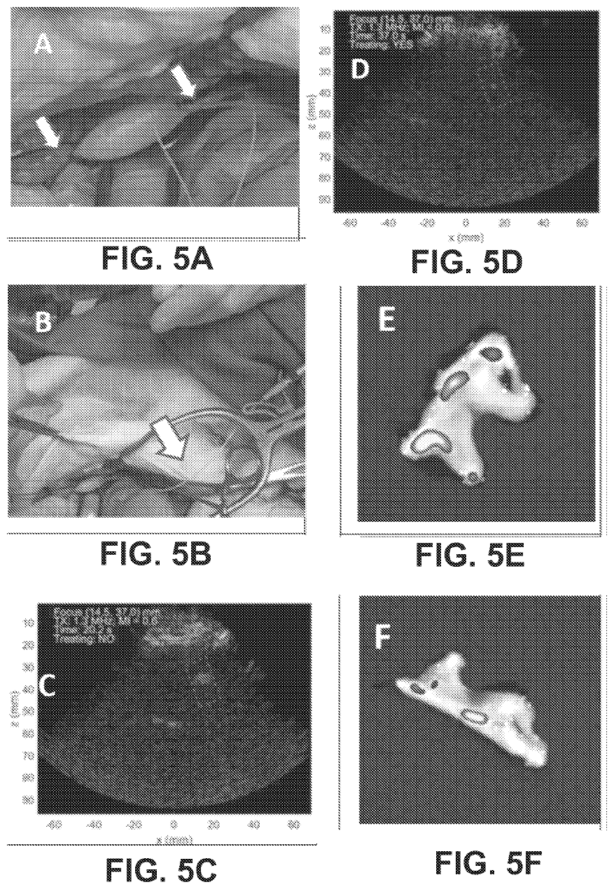

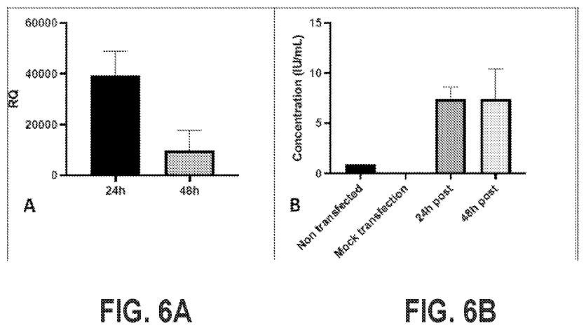

An example ultrasound system for theragnosis of vascular conditions is shown at FIG. 1. Herein, the ultrasound system is configured to image one or more blood vessels in a region of interest, and detect vascular conditions, such as occlusions (e.g., thrombus and/or embolus) in the one or more blood vessels (e.g., deep vein). Further the ultrasound system is configured to treat or ameliorate the detected vascular conditions of the one or more blood vessels or provide a preventative treatment to the region of interest. In one example, the ultrasound system is configured to treat, ameliorate, or prevent a vascular health condition by providing microbubble-based ultrasound-assisted gene therapy to induce local expression of one or more desired proteins (e.g., rtPA). A schematic illustration of an example method for theragnosis is shown at FIG. 2. An example method for imaging, diagnosis, and treating vascular conditions is shown at FIG. 3. Further, FIG. 4 shows an example method that may be performed using an example ultrasound theragnostic system, such as the system of FIG. 1. FIGS. 5A-5F show an example US-based transfection of porcine femoral vein. FIGS. 6A and 6B show example ex vivo expression of human tPA and its enzymatic activity in porcine MSCs.

### Example System

FIG. 1 illustrates a high-level block diagram of an ultrasound system 100, according to an embodiment of the disclosure. The ultrasound system 100 is configured to perform transcutaneous ultrasound imaging of one or more blood vessels of a subject, and process, in real-time or near real-time, the ultrasound data to output one or more reconstructed images of the one or more blood vessels. Further, the ultrasound system 100 is configured to provide transcutaneous ultrasound-based therapy to the subject. In one example, providing transcutaneous ultrasound-based therapy includes gene therapy to transfect vascular cells of the one or more blood vessels to induce localized expression of one or more desired proteins at and/or near a target region of interest.

The ultrasound system 100 comprises an ultrasound probe 102 (also referred to herein as “probe 102,” “transducer,” or “ultrasound transducer”) for acquiring ultrasound data, in real-time or near real-time.

The ultrasound system 100 further includes an ultrasound scanning unit 110 (also referred to as “scanning unit 110” or “scanner 110”) communicatively coupled to the probe 102, and a computing engine 140 communicatively coupled to the ultrasound scanning unit 110. Communication between the probe 102 and the scanning unit 110 may be wired, or wireless, or a combination thereof. Similarly, communication between the scanning unit 110 and the computing engine 140 may be wired, or wireless, or a combination thereof. While the present example shows the scanning unit 110 and the computing engine 140 separately, in some examples, the scanning unit 110 and the computing engine 140 may be configured as a single unit. Thus, the ultrasound data acquired via the probe 102 may be processed by an integrated/embedded processor within the ultrasound scanner 110. In some examples, the computing engine 140 and the scanning unit 110 may be separate but located within a common room. In some examples, the computing engine 140 may be located in a remote location from the scanning unit 110. For example, the computing engine 140 may operate in a cloud-based server that has a distinct and remote location with respect to other components of the ultrasound system 100, such as the probe 102 and scanning unit 110. Optionally, the scanning unit 110 and the computing engine 140 may be a unitary system that is capable of being moved (e.g., portably) from room to room. For example, the unitary system may include wheels or be transported on a cart. Further, in some examples, the probe 102 may include an integrated scanning unit and/or a computing engine 140, and as such, the ultrasound signal processing may be performed via the probe 102, and the processed signals may be transmitted (e.g., wirelessly and/or wired) directly to an output device, such as a display device 160.

In the illustrated embodiment, the ultrasound system 100 includes a transmitting unit 112 that drives an array of transducer elements (not shown) of the probe 102. The transducer elements may comprise piezoelectric crystals (or semiconductor based transducer elements) within the probe 102 to emit pulsed ultrasonic signals transcutaneously into one or more blood vessels of the subject. In one example, the transducer elements may be controlled via a transmit (Tx) sequencing and beamforming unit 116 housed in the scanning unit. For example, the Tx sequencing and beamforming unit may set a desired pulse pattern to be sent through each array element. Further, the transmitting unit 112 may comprise a plurality of pulser amplifiers 114 configured to drive individual array element based on the desired pulse pattern. In some examples, the transmitting unit 112 may include its own power module for supplying the required electrical voltages. In some examples, these electronic components (the sequencing and beamforming unit 116, the pulser amplifiers 114, and power modules) may be housed within a multi-layer printed circuit board (PCB) and/or implemented using integrated circuit (IC) chips. In some examples, the pulser amplifiers 114 may be implemented by using digital to analog converters with linear power amplification or MOSFET-based switches. In some examples, the Tx sequencing and beamforming unit 116 may be implemented using an FPGA. In some other examples hardwired logic may be utilized for implementing the Tx sequencing and beamforming unit 116.

After the elements of the probe 102 emit pulsed ultrasonic signals into a body of the subject, the pulsed ultrasonic signals are back-scattered from structures within an interior of the body, like blood cells or muscular tissue, to produce echoes that return to the elements. The echoes are converted into electrical signals by the elements and the electrical signals are received by the receiving unit 118. The electrical signals are processed by a plurality of analog to digital converters (ADCs) 120 and digital signals representing the received echoes are passed then through the beamforming unit 116 that outputs ultrasound data. Additionally, in some examples, transducer elements may produce one or more ultrasonic pulses to form one or more transmit beams in accordance with the received echoes.

In one example, the probe 102 may be a two dimensional array probe, and may include an array of a number of transducer elements. The number of transducer elements may be 64, 128, 256, 512, 1024 or other number suitable for ultrasound imaging of the blood vessels. As an example, an ultrasound theragnostic transducer (also referred to herein as US theragnostic transducer, or theragnostic transducer) configured for imaging and therapy of one or more blood vessels is provided, as discussed below. For example, ultrasound may be applied transcutaneously by placing the probe 102 on a subject's skin 104 to image and/or provide gene therapy to the one of more blood vessels. In the illustrated example, a vein 103 including valves 105 has a thrombus 106 and an embolus 107. During therapy, microbubbles 108 and plasmid deoxyribonucleic acid (DNA) 109 are injected at or near the treatment site. The ultrasound system 100 including the probe 102 is configured to image the vein 103, the thrombus 106, the embolus 107, and the microbubbles 108 and provide effective and safe ultrasound-based gene therapy for removing or reducing the occlusions (that is, the thrombus 106 and/or the embolus 107) in the vein by inducing localized and/or targeted expression of one or more therapeutic agents at the region of interest. The ultrasound system may be configured to image one or more blood vessels at greater depths from the skin surface and greater field of view, while also providing therapeutic transmissions for gene transfection only (and without affecting the thrombus and/or embolus) at these depths. As a result, treatment time is significantly reduced (e.g., from hours or days in intensive care with previous approaches for using catheter-based ultrasound to dissolve thrombus and/or embolus to few minutes (e.g., 15-45 minutes) for effective microbubble-assisted gene transfection to induce localized expression of one or more therapeutic agents). Further, the ultrasound parameters including mechanical index, frequency, and duration for gene transfection is safer for target and surrounding tissues compared to high-intensity and/or low-intensity focused ultrasound that previous approaches use for dissolving or breaking up thrombus and/or embolus. Furthermore, by employing ultrasound-assisted gene therapy, the thrombus and/or embolus is more effectively reduced thereby improving patient outcomes.

Further, the ultrasonic pulses emitted by the transducer elements of the probe 102 are back-scattered from structures in the body, for example, blood vessels and surrounding tissue, to produce echoes that return to the transducer elements. In one example, the probe 102 may be configured to transmit a plurality of plane waves from a selected number of element of the probe. Further, the plane waves may be steered over a range of angles (e.g., ±20° at a predetermined step size in one or more directions (e.g., lateral, azimuth, and/or elevation). In some examples, the plane waves may be steered at different range of angles in different directions (e.g., ±20° in azimuth and ±15° in elevation) to obtain a volumetric scan of the region of interest.

The echoes are received by the receiving unit 118. The received echoes are provided to a transmitter sequencing and beamforming unit 116 that performs beamforming and outputs an RF signal. The RF signal is then processed by the beamforming unit 116 (when implemented using an FGPA) or provided to a processor (not shown) of the scanning unit 110 (also referred to as a controller of the scanning unit 110) or a processor 142 of the computing engine 140 (also referred to as a controller of the computing engine 140) for processing the RF signal. Alternatively, the beamforming unit 116 or the processor may include a complex demodulator (not shown) that demodulates the RF signal to form IQ data pairs representative of the echo signals. In some examples, the RF or IQ signal data may then be provided directly to a non-transitory memory (not shown) for storage (for example, temporary storage).

In some examples, in order to detect blood flow, Doppler ultrasound imaging may be performed. Doppler ultrasound imaging detects movement of red blood cells by repeating ultrasonic pulses and evaluating temporal variations of successive backscattered signals. In one embodiment, ultrafast ultrasound imaging may be utilized based on plane wave emission for imaging blood flow changes. Plane wave emission involves simultaneously exciting all transducer elements of the probe 102 to generate a plane wave. Accordingly, the ultrasound imaging includes emitting a set of plane waves at titled angles in a desired range from a start degree to a final degree tilt of the probe 102 at a desired angular increment (e.g., 1 degree, 2 degrees, 3 degrees, etc.). An example desired range may be from −15 degrees to 15 degrees. In some examples, the desired range may be from approximately −30 degrees to +30 degrees. The above examples of ranges are for illustration, and any desired range may be implemented based on one or more of area, depth, and imaging system configurations. In some examples, an expected blood flow velocity may be considered in determining the desired range for imaging.

Further, in some examples, a 3-dimensional (3D) US sequence may be utilized for imaging one or more desired areas of a region of interest comprising one or more blood vessels. In one example, in order to acquire 3D US sequences, a plurality of linear scans may be performed while moving the probe to successive planes to perform a US acquisition at each position to generate 3D imaging data. In another example, in order to acquire 3D US sequences, a 2D matrix array or row-column array probe may be utilized to acquire 3D imaging data in a synchronous manner, i.e. without moving the probe. 3D imaging data thus obtained may be processed for evaluating hemodynamic activity and blood flow obstructions (e.g., thrombus, embolus, etc.) in the targeted areas of the body. Thus, the systems and methods described herein for may also be implemented by using 3D US imaging data without departing from the scope of the disclosure.

Imaging data from each angle is collected via the receiving unit 118. The backscattered signals from every point of the imaging plane are collected. In one example, the backscattered signals may be provided to the sequencing and beamforming unit 116 that performs a parallel beamforming procedure to output a corresponding RF signal. The RF signal may then be utilized by the processor, as discussed above, to generate corresponding ultrasonic image frames for each plane wave emission. Thus, a plurality of ultrasonic images may be obtained from the set of plane wave emissions. A total number of the plurality of ultrasonic images is based on acquisition time, a total number of angles, and pulse repetition frequency. In some examples, as shown, the receiving unit 118 may comprise the plurality of analog to digital converters 120 and low-noise amplifiers.

The plurality of ultrasonic images obtained from the set of plane wave emissions may then be added coherently to generate a high-contrast compound image. In one example, coherent compounding includes performing a virtual synthetic refocusing by combining the backscattered echoes of the set of plane wave emissions. Alternatively, the complex demodulator (not shown) may demodulate the RF signal to form IQ data representative of the echo signals. A set of IQ demodulated images may be obtained from the IQ data. The set of IQ demodulated may then be coherently summed to generate the high-contrast compound image. In some examples, the RF or IQ signal data may then be provided to the memory for storage (for example, temporary storage).

The computing engine 140 includes a processor 142 configured to process data from the Tx sequencing and beamforming unit 116. For example, the processor 142 may include an image-processing module that receives image data (e.g., ultrasound signals in the form of RF signal data or IQ data pairs) and processes image data. For example, the image-processing module may process the ultrasound signals to generate volumes or frames of ultrasound information (e.g., ultrasound images) for displaying to the operator. In the ultrasound system 100, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, contrast pulse sequence (CPS), C-scan, and elastography. The generated ultrasound images may be two-dimensional (2D) or three-dimensional (3D). When multiple two-dimensional (2D) images are obtained, the image-processing module may also be configured to stabilize or register the images.

Further, acquired ultrasound information may be processed in real-time or near real-time during an imaging session (or scanning session) as the echo signals are received. In some examples, an image memory may be included for storing processed slices of acquired ultrasound information that may be accessed at a later time. The image memory may comprise any known data storage medium, for example, a permanent storage medium, removable storage medium, and the like. Additionally, the image memory may be a non-transitory storage medium.

In operation, an ultrasound system may acquire data, for example, volumetric data sets by various techniques (for example, 3D scanning, real-time 3D imaging, volume scanning, 2D scanning with probes having positioning sensors, scanning using 2D or matrix array probes, and the like). In some examples, the ultrasound images using the ultrasound system 100 may be generated, via the processor 142, from the acquired data, and displayed to an operator or user via a display device 160 of a user interface 119, which may be communicatively coupled to the scanning unit 110 and/or the computing engine 140.

In some examples, the processor 142 is operably connected to the user interface 119 that enables an operator to control at least some of the operations of the ultrasound system 100. The user interface 119 may include hardware, firmware, software, or a combination thereof that enables a user (e.g., an operator) to directly or indirectly control operation of the ultrasound system 100 and the various components thereof. The user interface 119 may include the display device 160 having a display area (not shown). In some embodiments, the user interface 119 may also include one or more input devices (not shown), such as a physical keyboard, mouse, and/or touchpad. In an exemplary embodiment, the display device 160 is a touch-sensitive display (e.g., touchscreen) that can detect a presence of a touch from the operator on the display area and can also identify a location of the touch in the display area. The display device also communicates information from the processor 142 to the operator by displaying the information to the operator. The display device may be configured to present information to the operator during one or more of an imaging session and therapy session. The information presented may include ultrasound images, graphical elements, and user-selectable elements, for example.

Non-transitory memory 144 may further store a therapy module 145, which includes instructions for acquiring images during a therapy mode and performing ultrasound therapy. Therapy module 145 may include instructions that, when executed by processor 142, cause ultrasound system to generate therapeutic transmissions for imaging the plurality of microbubbles and/or performing gene therapy for transfection of one or more cells at or near the region of interest to induce localized protein expression of a therapeutic agent for treating a vascular condition.

According to some embodiments, the probe 102 may contain electronic circuitry to do all or part of the transmit beamforming and/or the receive beamforming. For example, all or part of one or more of the transmit/receive switch 122, the transmitting unit 112, the receiving unit 118, and the receiving unit 118 may be situated within the probe 102. The terms “scan” or “scanning” may also be used in this disclosure to refer to acquiring data through the process of transmitting and receiving ultrasonic signals. The term “data” may be used in this disclosure to refer to either one or more datasets acquired with an ultrasound system, such as the ultrasound system 100. In some embodiments, data acquired via the ultrasound system 100 may be used to train a machine learning model and/or be processed by the trained machine learning model to automatically detect one or more vascular health conditions discussed herein.

A user interface 119 may be used to control operation of the ultrasound system 100, including to control the input of patient data (e.g., patient medical history), to change a scanning or display parameter, to initiate a probe repolarization sequence, and the like. The user interface 110 may include one or more of the following: a rotary element, a mouse, a keyboard, a trackball, hard keys linked to specific actions, soft keys that may be configured to control different functions, and a graphical user interface displayed on a display device 160. In some embodiments, the user interface may be a user input device, and may comprise one or more of a touchscreen, a keyboard, a mouse, a trackpad, a motion sensing camera, an eye tracking camera, and other device configured to enable a user to interact with and manipulate data within the computing engine 140 and/or the scanning unit 110.

The computing engine 140 comprises a processor 142 configured to control the one or more components of the scanning unit 110 and/or the probe 102. In one example, the processer 142 is in electronic communication (e.g., communicatively connected) with the probe 102. For purposes of this disclosure, the term “electronic communication” may be defined to include both wired and/or wireless communications. The processor 142 may control the probe 102 to acquire data according to instructions stored on a memory of the processor 142, and/or memory 144. The processor 142 may control which of the elements of the probe 102 are active and the shape of a beam emitted from the probe 102. The processor 142 is also in electronic communication with the user interface 119, and the processor 142 may process the ultrasound data received from the transmit sequencing and beamforming unit 116 into images for display on the display device 160. The processor 142 may include a central processor (CPU), according to an embodiment. According to other embodiments, the processor 142 may include other electronic components capable of carrying out processing functions, such as a digital signal processor, a field-programmable gate array (FPGA), or a graphic board. According to other embodiments, the processor 142 may include multiple electronic components capable of carrying out processing functions. For example, the processor 142 may include two or more electronic components selected from a list of electronic components including: a central processor, a digital signal processor, a field-programmable gate array, and a graphic board. According to another embodiment, the processor 142 may also include a complex demodulator (not shown) that demodulates the RF data and generates raw data. In another embodiment, the demodulation can be carried out earlier in the processing chain. The processor 142 is adapted to perform one or more processing operations according to a plurality of selectable ultrasound modalities on the data. In one example, the data may be processed in real-time during a scanning session as the echo signals are received by the receiving unit 118, and transmitted to the beamforming unit 116 and/or the processor 142. The data may be stored temporarily in a buffer (not shown) during a scanning session and processed in less than real-time in a live or off-line operation. Some embodiments of the system may include multiple processors (not shown) to handle the processing tasks that are handled by processor 142 according to the exemplary embodiment described hereinabove. For example, a first processor may be utilized to demodulate and decimate the RF signal while a second processor may be used to further process the data, for example by augmenting the data as described in detail herein, prior to displaying an image. It should be appreciated that other embodiments may use a different arrangement of processors.

The memory 144 is included for storing processed frames of acquired data. In an exemplary embodiment, the memory 144 is of sufficient capacity to store at least several seconds' worth of frames of ultrasound data. The frames of data are stored in a manner to facilitate retrieval thereof according to its order or time of acquisition. The memory 144 may comprise any known data storage medium.

In various embodiments of the present system, data may be processed in different mode-related modules by the processor 142 (e.g., B-mode, contrast pulse sequence (CPS) mode, Color Doppler, M-mode, Color M-mode, spectral Doppler, Elastography, tissue velocity imaging (TVI), strain, strain rate, and the like) to form 2D or 3D data. For example, one or more modules may generate B-mode, CPS mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging and/or therapy) is being performed on a patient. The video processor module may include a separate image memory, and the ultrasound images may be written to the image memory in order to be read and displayed by display device 160.

In various embodiments, one or more components of ultrasound system 100 may be included in a portable, handheld ultrasound imaging device. For example, the user interface 119 may be integrated into an exterior surface of the handheld ultrasound imaging device, which may further contain computing engine 140. Probe 102 may comprise a handheld probe in electronic communication with the handheld ultrasound imaging device to collect raw ultrasound data. The components of the scanning unit 110 may be included in the same or different portions of the ultrasound system 100. For example, transmitting unit 112, the receiving unit 118, the switch 122, and the sequencing and beamforming unit 116 may be distributed between the handheld ultrasound imaging device, the probe, and combinations thereof.

According to some embodiments, computing engine 140 may be utilized for executing the entire signal processing chain using data from the sequencing and beamforming unit 116 as input. In some embodiments, the computing engine 140 may be configured as a high-end personal computer (PC) workstation. During operation, the incoming ultrasound data is fed from the front-end hardware, that is the scanning unit 110. Since this incoming data traffic is on the order of GB in size every second, the workstation to be equipped with sufficient computing resources to handle such a large data volume. While it is possible to perform processing by leveraging an on-board central processing unit (CPU), its processing capacity may be limited by the CPU's clock speed. In some examples, a parallel computing engine 150 comprising one or more GPUs 152 may be leveraged to facilitate high-throughput parallel processing of raw data samples. In some examples, multiple GPU devices may be connected to the workstation to scale the computing capacity. In one example, using GPU processing, delay-and-sum beamforming may be achieved at real-time throughputs. Other GPU-based beamforming algorithms have, such as spatial coherence imaging and minimum variance apodization, may be used. In some embodiments, various post-beamforming signal processing operations may also be performed using the GPU, such as Doppler imaging and related adaptive clutter filtering operations, motion estimation, temperature mapping for therapeutic monitoring, and image filtering. Further, in some examples, different GPU processing modules may be integrated to realize more advanced algorithms like high frame rate vector flow estimation and color encoded speckle imaging that may be integrated with a software-based open platform front-end to achieve live imaging of arterial and venous flow dynamics.

Turning next to FIG. 2, it shows a schematic illustration of an example method for theragnosis, treating, ameliorating, or preventing/reducing the likelihood of vascular diseases or disorders, such as DVT, PE, VTE, post-thrombotic syndrome, embolic strokes, embolic heart attacks, and combinations thereof. For example, the methods described herein may be implemented for recanalizing an occluded blood vessel by targeted transfection using ultrasound for expression or overexpression of a gene encoding for a therapeutic agent (e.g., fibrinolytic enzyme). The methods described herein may improve ultrasound-based gene therapy at regions of interest at various depths below the skin surface, thereby enabling transcutaneous ultrasound-based treatment in a minimally invasive or non-invasive manner. Further, the methods described herein may improve B-mode imaging and/or contrast pulse sequence imaging of a volumetric region of interest while providing therapeutic ultrasound for transfection without causing ultrasound induced damage at or near the volumetric region of interest. In some embodiments, the methods described herein may be used to reduce the risk of DVT, PE, VTE, post-thrombotic syndrome, embolic strokes, embolic heart attacks, and combinations thereof, via ultrasound-guided ultrasound-based gene therapy.

FIG. 2 shows features similar to those described at FIG. 1. Thus, similar features are similarly numbered. At 202, a deep vein thrombosis and embolus condition at the vein 103 is shown. In particular, the vein 103 comprises valves 105 and includes thrombus 106 and embolus 107 that has broken off from the thrombus 106 and travelling through the vein 103. The direction of blood flow is depicted by arrows 203. Further, at 202, an imaging ultrasound via a probe 201 of an ultrasound system, such as ultrasound system 100, may be performed to image the vein and the associated vascular disease (that is, thrombus and embolus in this example). The probe 201 may be configured to perform volumetric imaging of the vein 103 and one or more associated occlusions, such as a blood clot, thrombus 106, embolus 107, etc. The probe 201 is communicatively coupled to a scanning unit (not shown), such as scanning unit 110 and/or a computing engine (not shown), such as the computing engine 140. The probe 201 may be an example of probe 102 discussed with respect to FIG. 1. Thus, the probe 201 may be configured for imaging one or more of the blood vessels and its associated occlusion (e.g., anechoic areas such as thrombus, embolus, etc.). For example, the probe 201 may be configured to scan volumetric regions of interest over a depth range between 1-20 cm with a field-of-view up to 15 cm in azimuth and 11 cm in elevation. Further, the ultrasound data acquired via the probe 102 may be used to generate three dimensional images of the volumetric region of interest in real-time or near real-time. For example, an ultrasound system comprising the probe, the scanning unit, and the computing engine may be configured to generate three dimensional images using acquired ultrasound data at a desired volumetric imaging rate (e.g.,40 Hz to 60 Hz). The three dimensional rendering of the region of interest may be displayed on a display device, such as display device 160, communicatively coupled to one or more of the probe 201, the scanning unit, and the computing engine.

At 202, the probe 201 may be operated in an imaging mode for imaging the vein 103 and one or more of thrombus 106 and embolus 107. In some examples, the imaging may be performed immediately before the administration of a composition for ultrasound-assisted gene therapy, as discussed further below. As a result, imaging and diagnosis may be immediately followed by therapy, which may improve patient outcomes.

Next, at 204, plasmid DNA 109 including the gene encoding for the therapeutic agent (e.g., fibrinolytic enzyme) and mixed with an ultrasound contrast agent comprising microbubbles is injected into the vein 103 at or near the region of interest comprising one or more of the thrombus 106 and embolus 107. Accordingly, in one example, a composition for treating, ameliorating, or preventing/reducing the likelihood of a vascular condition may comprise an ultrasound contrast agent and a vector for gene transfection, the vector including at least the gene encoding the therapeutic agent. Further, the ultrasound contrast agent may comprise a plurality of microbubbles. Alternatively, in some examples, the ultrasound contrast agent may comprise a plurality of nanobubbles or a combination of microbubbles and nanobubbles.

Next, at 206, transcutaneous ultrasound is applied via probe 201 in a therapeutic mode for facilitating transfection of the injected plasmid into the vein 103. In particular, the therapeutic mode may be used for transfecting vascular cells, such as endothelial cells forming the inner lining of the blood vessels at the luminal side of the vein, muscle cells of tunica media, and/or adventitial cells of tunica adventitia at the adventitial side of the vein, with plasmid including one or more of a desired gene (e.g., rtPA gene) and one or more reporter genes. In some examples, the therapeutic mode may also enable imaging of microbubbles. In some examples, a second imaging transmission for imaging of the ultrasound contrast agent, different from the first imaging transmission for imaging the vein and its associated occlusion, may be performed, and a therapeutic emission for transfection of injected plasmid DNA may be performed. In some embodiment, the second imaging transmission and the therapeutic emission may be alternated until a desired distribution of microbubble is achieved (e.g., when a number of visible microbubbles in the region of interest is less than a threshold), which may indicate an end point for therapy.

In this way, ultrasound-guided ultrasound-assisted gene therapy may be performed for treatment, amelioration or prevention of a vascular disease.

Further, step 208 indicates expression of the therapeutic agent 111 at or near the site of interest following successful transfection of the gene encoding the therapeutic agent. In one example, the therapeutic agent may be selected from the group consisting of tissue plasminogen activator, recombinant tissue plasminogen factor, urokinase, plasminogen, and streptokinase. In some example, one or more therapeutic agents may be used, where the one or more therapeutic agents are selected from the group consisting of tissue plasminogen activator, recombinant tissue plasminogen factor, urokinase, plasminogen, streptokinase, and any combination thereof. Accordingly, the vector used for transfection may include one or more gene sequences encoding for the one or more therapeutic agents.

The therapeutic agent 111 interacts with the thrombus 106 and the embolus 107, which results in dissolution of the thrombus 106 and the embolus 107. Accordingly, the illustration at 210 indicates complete dissolution of the thrombus and the embolus, and re-canalization of the obstructed vein 103.

Next, FIG. 3 shows a flow chart illustrating a high-level method 300 for performing ultrasound-guided ultrasound assisted gene therapy for treating, ameliorating, or preventing/reducing the likelihood of a vascular condition of a subject.

Step 302 of the method 300 includes providing an ultrasound system, such as the ultrasound system 100 at FIG. 1, comprising an ultrasound probe, such as the probe 102, and one or more processors, such as processor 142 of the computing engine 140. The method 300 will be described with respect to the ultrasound system 100 at FIG. 1; however, it will be appreciated that the method 300 may be implemented using similar systems without departing from the scope of the disclosure.

In some implementations, step 302 of the method 300 further includes imaging one or more blood vessels via the probe using a first imaging transmission. In particular, the probe is operated to generate the first imaging transmission to generate first ultrasound data of the one or more blood vessels and any associated occlusions in the region of interest. Further the first ultrasound data is used to obtain B-mode image data for imaging the one or more blood vessels and any associated occlusions. The first imaging transmission includes a first set of plane waves generated by steering from a first angle to a second angle at a first predetermined degree increment in a first direction (e.g., azimuth) and further includes a second set of plane waves generated by steering from a third angle to a fourth angle at a second predetermined degree increment in a second direction (e.g., elevation) for acquiring ultrasound data of a region of interest. As discussed above, the configuration of the transducer wherein a first number of elements are separated by a first pitch in the first direction (e.g., azimuth), and wherein a second number of elements are separated by a second pitch different from the first pitch in the second direction (e.g., elevation) advantageously provides an extended field of view at greater depths for imaging the one or more blood vessels and any associated occlusions transcutaneously. Further, B-mode imaging for detecting blood vessels and associated occlusions is performed at a first transmitting frequency greater than a second frequency used for therapeutic imaging. In one example, the first transmitting frequency may be 2.5 MHz.

Step 304 of the method 300 includes identifying one or more occlusions in the blood vessels based on three dimensional reconstructions of the one or more blood vessels and/or associated occlusions (e.g., thrombus, embolus, etc.) in the region of interest. In some examples, method 300 include step 306 where, upon identifying one or more blood vessels and associated occlusions, a first blood flow velocity may be determined in the region of interest prior to performing ultrasound-based gene therapy. For example, Doppler longitudinal and transverse sections may be generated, as well as pulsed Doppler may be employed, to measure blood flow velocity.

Step 308 the method 300 includes administering an ultrasound contrast agent for performing ultrasound-based gene therapy. In one example, the ultrasound contrast agent comprises a solution including microbubbles and a DNA vector, such as plasmid DNA including one or more genes that encode one or more proteins involved in recanalization of occluded blood vessels and dissolution of occlusions (e.g., thrombus, embolus). In various embodiments, the microbubbles may be gas-filled microbubbles (e.g. 3-10 μm diameter) stabilized by a flexible shell such as phospholipids or albumin. In an ultrasound field the microbubbles generate nonlinear scattered signals, which enables discrimination of the blood flow from surrounding tissue. Non-limiting examples include perfluorobutane microbubbles and sulfur hexafluoride microbubbles. Commercially available ultrasound contrast agents that can be used with the systems and methods described herein, and include, by way of example: Targesphere® and Targesphere® SA (available from Targeson, San Diego, Calif.; see Tlaxa et al. (2010) Ultrasound Med. Biol. 36(11):1907-18); Optison® (GE Healthcare), albumin microbubbles with an octafluoropropane gas core; Levovist® with (Schering), having a lipid/galactose shell and a core of air; Imagent® lipid microspheres with a perflexane core; Definity® lipid microspheres with an octafluoropropane gas core; Lumason® sulfur hexafluoride lipid microbubbles (previously Sonovue®), MicroMarker microbubbles (Bracco Imaging S.p.A./Fujifilm Visualsonics), EchoGen® having dodecafluoropentane emulsion, Albunex® having 5% human albumin, Myomap™, and Perfluorocarbon containing microbubbles (Perfluorocarbon exposed sonicated dextrose albumin PESDA). In some examples, the ultrasound contrast agent comprises only a suspension of microbubbles and plasmid DNA.

In one example, the ultrasound contrast agent comprising microbubbles and the DNA vector may be injected into the one or more blood vessels at or near the region of interest for performing gene therapy.

In various embodiments, the microbubbles may be modified to target specific cell type and/or to carry the vector DNA, as discussed further below. For example, the microbubbles may be modified with targeting antibodies that recognize specific cell types in order to increase transfection of the specific cell types. Additionally, in some examples, the microbubbles may be modified to carry vector DNA in order to achieve improved gene transfection in the specific cell types.

In some examples, the microbubbles are in a size range between 0.5 μm and 15 μm.

While the above examples describe microbubbles as contrast agents and for ultrasound-assisted gene therapy for vascular conditions, in some examples, nanobubbles may be bubbles may be used. Accordingly, in various embodiments, the nanobubbles may be modified to target specific cell type and/or to carry the vector DNA. For example, the nanobubbles may be modified with targeting antibodies that recognize specific cell types in order to increase transfection of the specific cell types. Additionally, in some examples, the nanobubbles may be modified to carry vector DNA in order to achieve improved gene transfection in the specific cell types.

Step 310 of the method 300 includes imaging the injected microbubbles via the probe using a second imaging transmission. The second imaging transmission comprises a contrast pulse sequence (CPS) for each plane wave transmitted. In one example, pulse inversion and pulse modulation is combined, which in some examples comprises transmitting three pulses ½, −1, ½. The coherent summation of the echoes from each excitation resulted in a CPS signal in which the linear oscillation has been removed. The plane wave sequence may be similar to the first imaging transmission including the first set of plane waves generated by steering from the first angle to the second angle at the first predetermined degree increment in a first direction (e.g., azimuth) and further includes the second set of plane waves generated by steering from the third angle to the fourth angle at the second predetermined degree increment in the second direction (e.g., elevation) for acquiring ultrasound data of the region of interest. Further, a second transmission frequency may be used to image the microbubbles. In this way, the CPS mode is used to image non-linear oscillation of the microbubbles.

Step 312 of the method 300 includes providing therapeutic ultrasound pulse for transfection of the plasmid DNA into vascular cells of the one or more blood vessels. In one example, the therapeutic pulse comprises 1.3 MHz and Mechanical Index of 0.6. In some examples, therapeutic pulse and the second imaging sequence may be alternated to visualize microbubbles while providing the therapeutic pulse. Further, by monitoring microbubbles during therapy, a timing of therapeutic pulse may be adjusted based on the distribution and amount of microbubbles in the volume of interest. In one example, as indicated at step 314, the method 300 can include, responsive to a number of microbubbles in a volumetric region of interest decreasing below a threshold number, the therapeutic pulse may be stopped.

In this way, diagnosis and gene therapy may be effectively provided to blood vessels located at various regions of interest from the skin surface.

In some examples, a vascular heath condition after providing gene therapy may be monitored periodically (e.g., the treatment site may be monitored every four days). Further, the region of interest may be re-treated if desired progress (e.g., reduction in thrombus size, dissolution of thrombus, etc.). The re-treatment may be performed as discussed above at FIG. 3. During the monitoring, one or more properties of one or more occlusions of the blood vessels may be evaluated. This includes monitoring reduction in size and/or position of the occlusion. For example, a 2D probe, such as probe 102, may be used to image the one or more occlusions and based on comparison of the 3D reconstructed images obtained before and after a period of time (e.g., 1, 2, 3, 4, 5, 6, or 7 days) following the gene therapy, a change in size of the occlusion may be evaluated. Further, a change in blood flow may be evaluated using Doppler imaging. Further, in some examples, an expression of an in vivo marker may be monitored.

If a desired reduction in occlusion size is not achieved and/or if a desired improvement in blood flow velocity is not achieved, the region of interest may be re-treated.

### Target Cells and Microbubble Loading/Modifications

In some embodiments, targeted gene therapy may be provided for treatment of vascular diseases and conditions. As one example, endothelial progenitor cells may be recruited into a thrombus. Inventors have recognized that targeted transfection of the endothelial progenitor cells with a gene encoding for a therapeutic agent (e.g., tissue plasminogen activator, recombinant tissue plasminogen factor, urokinase, plasminogen, or streptokinase) may accelerate thrombus resolution. Accordingly, microbubbles coated with targeting antibodies (e.g., targeting antibodies conjugated with a shell of a microbubble) may be mixed with a vector carrying a gene encoding the therapeutic agent. Targeting antibodies refer to antibodies that are specific for antigens (e.g., cell surface markers) expressed or overexpressed in a specific cell type (e.g., endothelial progenitor cells). Targeting microbubbles refer to microbubbles that include targeting ligands on the microbubble shell. In one example, the targeting ligands include targeting antibodies. Thus, in one example, a composition comprising a contrast solution comprising targeting microbubbles and a vector including a gene encoding a therapeutic agent for thrombus and/or embolus dissolution may be used for ultrasound-mediated gene therapy. The targeting microbubbles include a plurality of microbubbles, each of the plurality of microbubbles conjugated with one or more targeting antibodies, where the one or more targeting antibodies recognize a cell surface marker (e.g., vascular endothelial growth factor-2, CD133, CD34) of a target cell (e.g., endothelial progenitor cells). In this way, the targeting microbubbles including the targeting antibodies are recruited to the target cells. When therapeutic pulse is applied, the targeting microbubbles increase transfection rate of the target cells (e.g., by increasing permeability of the target cells), thereby enabling transfection of the target cells with the vector including the gene for the therapeutic agent. In some examples, targeting antibodies may be tethered to the microbubbles via linkers, such as Fc-region-binding polypeptides derived from protein A/G, PEG, etc.

In some examples, the target cells may be endothelial cells, endothelial progenitor cells, smooth muscle cells, or any combinations thereof. In some embodiments, these cells are endogenous cells. Accordingly, in some examples, microbubbles used in the methods described herein may be modified with antibodies that recognize cell surface markers of one or more cell types, the one or more cell types including one or more of endothelial cells, endothelial progenitor cells, and smooth muscle cells.

In some examples, additionally or alternatively to conjugating the microbubbles with the targeting antibodies (that is, antibodies targeting a specific cell type), vector encoding the therapeutic agent may be attached to the targeting microbubbles (e.g., via through use of electrostatic attractions). An example microbubble may comprise targeting antibodies and/or plasmid DNA encoding the therapeutic agent loaded onto the shell of the microbubble.

In some examples, targeting antibodies may be conjugated to a shell of a microbubble while the DNA vector may be encapsulated within the shell of the microbubble.

In some examples, targeting microbubbles may be optionally loaded with nucleic acids-liposome complexes (lipoplexes) or nucleic acids-polymer complexes (polyplexes).

In some examples, different sets of microbubbles, each set targeting a different cell type may be used. In some other examples, different sets of microbubbles, each targeting the same cell type but carrying different genes encoding different therapeutic agents may be used. In still further examples, a set of microbubbles may target multiple cell types and carry vector encoding a therapeutic agent. In yet another example, a set of microbubbles may carry a vector having a first gene for a first therapeutic agent and a second gene for a second therapeutic agent.

In another example, a first set of microbubbles targeting a first cell type and loaded with a first DNA vector encoding a first therapeutic agent, a second set of microbubbles targeting the first cell type and loaded with a second DNA vector encoding a second therapeutic agent, and so on may be used.

FIG. 4 shows a flow chart illustrating an example method 400 for performing ultrasound image guided microbubble-assisted gene therapy for treatment of vascular diseases. The method 400 may be executed by a processor, such as processor 142 or a processor of the scanning unit, such as scanning unit 110 or a combination thereof. The processor may be communicatively coupled to an ultrasound probe, such as probe 102, configured to image one or more of vascular features including one or more of blood vessels, occlusions in the blood vessels, blood flow, and microbubbles, transcutaneously at greater depths from the skin surface and/or with an extended field of view. The method 400 may be executed during image guided gene therapy for vascular diseases. The method 400 will be described below with respect to FIG. 1; however, it will be appreciated that the method 400 may be implemented by other similar systems without departing from the scope of the disclosure.

Step 402 of the method 400 includes acquiring ultrasound data of a region of interest based on a first imaging mode. For example, a user (e.g., a clinician, a health care provider) may indicate, via a user interface communicatively coupled to the probe, the first imaging mode. Responsive to the input for operating the probe in the first imaging mode, a first imaging ultrasound sequence may be transmitted via the probe to obtain ultrasound data of the region of interest. In one example, step 402 of the method 400 includes performing B-mode imaging 403. B-mode images are generated using the ultrasound data of the region of interest. In some examples, the images may be volumetric images. That is, the ultrasound data generated using the first imaging mode is used to reconstruct B-mode images of one or more blood vessels and/or any associated occlusions (e.g., thrombus, embolus, etc.) in the region of interest. Additionally or alternatively, 3D volumetric renderings of B-mode images of the region of interest may be generated. Further, in some examples, the one or more 2D B-mode renderings and/or 3D B-mode renderings may be displayed on a display device communicatively coupled to the probe and/or the processor at option step 404 of the method 400. Furthermore, the one or more 2D B-mode images and/or the 3D B-mode image may be generated and displayed in real-time or near real-time. In some examples, plane waves may be used for generating the ultrasound data. For example, by using plane waves, high imaging frame rate may be achieved that enables real-time or near real-time imaging of the blood vessels and any associated occlusions at deeper regions of interest below the skin surface.

Step 406 of the method 400 includes determining if ultrasound treatment is desired based on user input, for example. If the answer is NO, the method 400 may continue to step 408, where the region of interest can be imaged using the first imaging mode. For example, ultrasound B-mode imaging may be performed until an input is received from the user to terminate the imaging process. If the answer is YES, the method 400 proceeds to step 410.

Step 410 of the method 400 includes acquiring ultrasound data based on a second imaging mode to visualize the injected microbubbles. The second imaging mode may use a plane wave sequence of the first imaging mode. Accordingly, the second imaging mode may include transmitting the first set of plane waves from the first angle to the second angle at the first predetermined degree increment in the first direction (e.g., azimuth) and further includes transmitting the second set of plane waves from the third angle to the fourth angle at the second predetermined degree increment in a second direction (e.g., elevation) for acquiring ultrasound data of the region of interest. However, for each plane wave transmission a CPS may be employed. An example CPS may comprise three pulses (½, −1, ½) for each plane wave. In some examples, a number of CPS pulses for each plane wave may be 3, 4, or 5. Additionally, a second transmission frequency less than the first transmission frequency may be used in the second imaging mode for imaging the microbubbles within the blood vessels. An example transmission frequency may be in a range between 1.3 MHz and 1.5 MHz.

Step 412 of the method 400 includes processing the ultrasound data generated in the second imaging mode to generate, in real-time or near real-time, one or more 2D contrast images of the microbubbles in the blood vessels in the region of interest and/or 3D contrast images of the microbubbles. In some examples, the B-mode images and the corresponding contrast images may be displayed side-by-side. Additionally or alternatively, the contrast images may be overlaid on the B-mode images or vice-versa. Further, the type of display (e.g., contrast mode only, side-by-side, overlaid, etc.) may be user selectable and may be indicated by the user via the user interface.

Step 414 of the method 400 includes initiating a therapeutic pulse sequence. In one example, the therapeutic pulse sequence includes 1.3 MHz and Mechanical Index0.6. In one example, the second imaging sequence may be alternated with the imaging sequence, which enables monitoring of the microbubble behavior during therapy. The therapeutic sequence may be configured to transfect the DNA vector (e.g., plasmid DNA) that is injected with the contrast solution into the vascular cells of the subject in the region of interest to provide localized expression of one or more desired proteins (e.g., fibrinolytic factor) for reduction and/or dissolution of one or more occlusions and recanalization of any obstructed blood flow due in the blood vessels due to one or more occlusions.

Step 416 of the method 400 includes quantifying microbubble distribution and determining if an amount of microbubbles in a given area or volume of region of interest is less than a threshold. If YES, the method 400 proceeds to step 418 where the therapeutic transmission is automatically stopped. If the answer at step 416 is NO, the method 400 proceeds to step 420 and continues to provide therapeutic transmissions and second imaging transmissions until the amount of microbubbles is below the threshold. Optionally, the therapeutic transmission may be terminated based on user input. Further, in some examples, during the therapeutic transmission, a change in microbubble distribution may be determined and indicated via the display device to the user.

In this way, the systems and methods described herein are configured to image one or more of blood vessels, associated occlusions, microbubbles, and blood flow in the blood vessels to evaluate a vascular health condition and concurrently provide microbubble assisted gene therapy to treat, ameliorate, or prevent one or more vascular diseases. The imaging and treatment modes described herein provide effective theragnosis for various vascular conditions in a non-invasive or minimally invasive manner, thereby reducing the need for invasive treatments, such as catheter directed thrombolysis, or ineffective treatments, such as anticoagulants.

In one representation, provided herein is an ultrasound system comprising an ultrasound probe; a non-transitory memory having instructions stored therein, and a controller. The controller can execute the instructions to: during a first imaging mode, transmit, via the ultrasound probe, a first plane wave sequence at a first transmission frequency to image one or more blood vessels and/or associated occlusions in a volumetric region of interest; and during a second imaging mode, transmit, via the probe, a second plane wave sequence at a second transmission frequency to image a plurality of microbubbles within the one or more blood vessels in the volumetric region of interest

In one representation, provided herein is an ultrasound system comprising an ultrasound probe that includes an array of transducer elements; a non-transitory memory having instructions stored therein, and a controller. The controller can execute the instructions to: during a first imaging mode, acquire, via the ultrasound probe, a first set of ultrasound image data of one or more blood vessels and/or an associated occlusion in a volume of interest; and during a second imaging mode, acquire, via the ultrasound probe, a second set of ultrasound image data of a plurality of microbubbles in the one or more blood vessels and/or an associated occlusion in a volume of interest.

In some implementations, provided herein is use of an ultrasound system for theragnosis, treating, ameliorating, or preventing/reducing the likelihood one or more vascular conditions in a subject. The ultrasound system comprises an ultrasound probe; a non-transitory memory having instructions stored therein, and a controller. The controller can execute the instructions to: during an imaging mode, transmit, via the probe, an ultrasound plane wave sequence to image a plurality of microbubbles within the one or more blood vessels in the volumetric region of interest and during a therapy mode, transmit, via the probe a therapeutic ultrasound sequence for transfection of a gene therapy vector into one or more vascular cells; providing a therapeutic composition to the subject, the therapeutic composition comprising the plurality of microbubbles and the gene therapy vector comprising a gene encoding a therapeutic agent for the treatment, amelioration or prevention of the one or more vascular conditions; and providing the therapeutic ultrasound sequence.

In some implementations, provided herein is use of a therapeutic composition for ultrasound-assisted gene therapy, the therapeutic composition comprising an ultrasound contrast agent comprising microbubbles or nanobubbles, and a gene therapy vector including a gene encoding a therapeutic agent for dissolution of thrombosis and/or embolus in a blood vessel.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent trials, means or reactants without the exercise of inventive capacity and without departing from the scope of the invention.

### Example 1: Ultrasound Based Transfection of Pig Femoral Vein

In one example, transfection of a porcine femoral vein with a reporter gene using MB-enhanced, US-mediated gene delivery is shown at FIGS. 5A-5F. A minipig cadaver was used shortly following euthanasia in an unrelated study. The femoral vein was exposed, washed with sterile PBS, and tied with sutures on both ends (FIG. 5A). A catheter was inserted into the vein and 10 μL of Definity MBs mixed with 1 mg of GFP Plasmid (pCMV-GFP) were injected (FIG. 5B). US was transcutaneously applied in contrast mode (1.3 MHz, MI=0.6; using a Philips ATL P4-1 Phased Array US Transducer connected to a 256 Advantage Verasonics system). US was applied until MBs were no longer visible (FIGS. 5C and D), approximately 2 minutes. The vein segment was then removed and immediately transferred into culture media (RPMI) supplemented with 10% FBS. Forty-eight hours later, the vein was cut lengthwise and imaged using a fluorescence imaging system (IVIS). A strong fluorescent signal was evident from both the luminal and adventitial sides of the vein (FIGS. 5E and 5F).

Example 2: Overexpression of human tissue plasminogen activator (tPA) in porcine MSCs (pMSCs) in FIGS. 6A and 6B. Herein, MSCs were isolated from porcine bone marrow (pBM-MSCs), as previously described by Bauersachs et al in Impact of gender on the clinical presentation and diagnosis of deep-vein thrombosis. Thromb Haemost. 2010;103(4):710-7.,which is incorporated by reference herein in its entirety. Cells were transfected ex vivo using a nucleofector system (Lonza) buffer, and 10 μg of tPA plasmid (pcDNA3.1(+)-P2AeGFP). Twenty-four- and 48-hours post-transfection, RNA was extracted using a RNeasy Mini Kit (Qiagen). RNA was then reverse transcribed to cDNA using the High-Capacity cDNA Reverse Transcription Kit (Thermo Fisher) and subjected to quantitative RT-PCR (qRTPCR) using a TaqMan Gene Expression Assay. Results showed high expression 24 hours post-transfection, which was reduced after 48 hours (FIG. 6A). Results are shown in comparison to gene expression in a mock-transfected sample. In addition, culture media was collected from the transfected cells' plates at 24- and 48-hours post-transfection. Media was analyzed using the Tissue Type Plasminogen Activator Activity Assay Kit (Abcam). Optical density (OD) at 405 was used to quantify the concentration of active tPA. Results showed the presence of active tPA in the media of transfected cells compared to media collected from non-transfected and mocktransfected cells (FIG. 6B).

### Example 3: Development of An US Protocol for Imaging and Drug Delivery to Venous Thrombi

In one example, a 2D array may be used to image a thrombus and MBs injected into the thrombus, using an in vitro model. Next, a rodent thrombosis model can be used to evaluate intravenous transfection using reporter genes and MBs.

Study design: There are ample examples of in vitro thrombus models in the literature. Most of the models involve a flow-through system mimicking blood circulation, since the goal was to induce thrombolysis via mechanical disruption based on ultrasound. However, the goal is to use ultrasound for imaging and MB oscillation and therefore a simpler, static, model of a thrombus may be used, as described below. Next, ultrasound may be used for in vivo intravenous cell transfection. Rats with tail thrombosis may be injected with a mixture of reporter gene plasmids and MBs, followed by ultrasound application (Tables 2A and 2B below). The optimal MB dose and US parameter sets may be determined using Luciferase transfection. The extent and duration of transgene expression may be monitored using in vivo bioluminescence imaging. Next, the GFP reporter gene may be used to determine cell transfection percentage using flow cytometry and spatial expression in situ, using histology and immunofluorescence.

TABLE 2A
Experimental Groups Aim 1 - Luciferase transfection

| Groups | Model | Plasmid | MBs | US | # of animals |
|---|---|---|---|---|---|
| 1 | Tail thrombosis | Luciferase 50 μg | Dose A | 4 parameter sets | 24 (6/subgroup) |
| 2 | Tail thrombosis | Luciferase 50 μg | Dose B | 4 parameter sets | 24 (6/subgroup) |
| 3 | Tail thrombosis | Luciferase 50 μg | Dose A | — | 3 |
| 4 | Tail thrombosis | Luciferase 50 μg | Dose B | — | 3 |
| 5 | Intact tail | Luciferase 50 μg | Dose A | 4 parameter sets | 24 (6/subgroup) |
| 6 | Intact tail | Luciferase 50 μg | Dose B | 4 parameter sets | 24 (6/subgroup) |
| 7 | Tail thrombosis | — | — | — | 3 |
| 8 | Intact tail | — | — | — | 3 |

TABLE 2B
Experimental Groups Aim 1 - GFP transfection

| Groups | Model | Plasmid | MBs | US | # of animals |
|---|---|---|---|---|---|
| 9 | Tail thrombosis | GFP 50 μg | TBD | Yes (TBD) | 12 (flow cytometry + histology) |
| 10 | Intact tail | GFP 50 μg | TBD | Yes (TBD) | 12 (flow cytometry + histology) |
| 11 | Tail thrombosis | — | — | — | 3 (flow cytometry) |
| 12 | Intact tail | — | — | — | 3 (flow cytometry) |

### Materials & Methods:

Reagents: Plasmids encoding for eGFP and Luciferase under CMV promoter may be used. Additional reagents to be used may include MBs (Definity, Lantheus) and commercial molecular biology reagents indicated below.

In vitro thrombosis model: Blood clots may be prepared as described in Kim Het al. in A multi-pillar piezoelectric stack transducer for nanodroplet mediated intravascular sonothrombolysis. Ultrasonics. 2021;116:106520, which is incorporated herein by reference in its entirety. Fresh bovine blood (Densco Marketing Inc., Woodstock, IL) may be mixed with 2.75% (w/v) CaCl2 solution (Fisher Scientific, Fair Lawn, NJ) in a volume ratio of 10:1. The mixture may be loaded in Tygon tubes with inner diameter of 6.35 mm. Next, the Tygon tubes with the blood-CaCl2 solution may be placed inside a 37° C. water bath for three hours to induce coagulation. The coagulated blood may be stored at 4° C. for one week.

Imaging studies of in vitro thrombus model: B-mode and CPS imaging, followed by therapeutic pulse application may be applied to the thrombus model with or without MB addition.

In vivo thrombosis model: In order to test cell transfection in a thrombus model, an established rat model of tail thrombosis may be used that is based on hypercoagulopathy induced k-Carrageenan and stasis using ice cold water. Four-week-old female and male Wistar rats weighting 150-200 g may be included in this study. Under general anesthesia the rat tails may be ligated approximately 13 cm from the tip of the tail and k-Carrageenan (Sigma Aldrich) dissolved in saline at a 1 mg/ml concentration may be injected into the dorsal tail vein. After the injection, the tails may be soaked in ice-water bath (4° C.) for 1 min. The ligation may be removed after 10 minutes.

This protocol was shown to induce thrombosis in 95% of the animals within 6 hours, which lasts for 3 days. Animals that may not develop thrombosis may not be included in the study. In vivo intravenous transfection: Intravenous reporter gene transfection may be done described in Ilovitsh T et al in Low-frequency ultrasound-mediated cytokine transfection enhances T cell recruitment at local and distant tumor sites. Proc Natl Acad Sci U S A. 2020; 117(23):12674-85, which is incorporated by reference in its entirety. Luciferase or GFP plasmids (50 μg/injection) may be mixed with Definity MBs (2×106 or 2×107), and injected into the dorsal tail vein, 1 day after the induction of thrombosis. Animals with or without thrombosis, may be injected with Luciferase/MBs and treated or not with US (Groups 1-6). Control groups may not be injected with Luciferase/MB (Groups 7-8). All groups may be monitored for gene expression using bioluminescence imaging 24 hours, 3- and 7-days post transfection. An IVIS Spectrum system (Perkin Elmer, Waltham, MA, USA) may be used. Five minutes prior to the imaging, the rats may be injected with luciferin diluted with PBS+/+ (25 mg/injection; Promega, Madison, WI, USA), percutaneous at the transfection site. Ten minutes post luciferase injection, the rats may be placed in the IVIS machine under anesthesia with 2% isoflurane in oxygen (2 L/min). Bioluminescence may be quantified as the total flux in the area covered by the region of interest.

The MB dose and US parameter sets to use in the GFP-transfection groups (Groups 9-10; n=6 each for flow cytometry and histology) may be determined based on the bioluminescence imaging results. Control groups may untreated animals with or without thrombosis (Groups 11-12).

Ex vivo evaluation of transfection: GFP-transfected animals may be euthanized on Day 3 post transfection. In each group 6 tails may be subjected to flow cytometry and 6 may be processed for immunohistochemistry. Tails may be harvested, divided to three segments, and labeled according to proximity to injection site of the plasmid.

Single-cell suspensions may be obtained by removal of vertebral bones and mechanical disruption of the tissue with scissors followed by enzymatic digestion with 1 mg/mL collagenase IV (Sigma Aldrich, St. Louis, MO) for 60 min at 37° C. and filtration through a 70 μm cell strainer (BD Biosciences, San Jose, CA). All cell suspensions may be stained using the LIVE/DEAD® Fixable Aqua Dead Cell Stain Kit (Invitrogen, Carlsbad, CA) according to the manufacturer's instructions in order to exclude dead cells from analysis. All cell preparations may be fixed in Cytofix buffer (BD Biosciences) diluted to 1% paraformaldehyde (PFA) in PBS. Cells may be analyzed on an LSR II flow cytometer (BD, San Jose, CA), and all datasets may be analyzed using FlowJo software vX (TreeStar).

For immunohistochemistry, paraffin-embedded sections of tails may be prepared and used for the detection of GFP-expressing cells using anti-GFP antibody at 1/400 dilution (Alexa Fluor 555 conjugated, rabbit polyclonal IgG (indirect detection, cat#A-31851, ThermoFisher), as described by Sper et al. in Generation of a Stable Transgenic Swine Model Expressing a Porcine Histone 2B-eGFP Fusion Protein for Cell Tracking and Chromosome Dynamics Studies. PLOS One. 2017;12(1):e0169242, which is incorporated by reference herein in its entirety. Cells may be counterstained with DAPI. Stained sections may be examined using a confocal microscope to determine the location of transfected cells.

Statistical Considerations: Data may be tested across groups with factorial ANOVA. For luciferase studies of method optimization with a sample size of 6 per group, there will be 86% power to detect a 4×10⁴ difference in MB dose and a 5×10⁴ difference US parameters based on prior studies where SD=1×10⁵ was observed.

For GFP studies, with 6 per group for analysis, there will be 80% power to detect the expected differences of >15% in transfection rates between treated and control groups with standard deviation of 4%. Tukey's posthoc testing may be used to correct for multiple testing at the two-sided alpha of 0.05.

### Example 4: In Vivo US-Mediated Transfection in a Porcine DVT Model

Catheter-directed thrombolysis using rtPA has several pitfalls, including long infusion time; need for intensive monitoring; long hospital stay; and risk of intracranial bleeding. Local gene expression of tPA may offer a better therapeutic solution, overcoming these disadvantages. Furthermore, endothelial progenitor cells (EPCs) play a pivotal role in the process of DVT thrombus resolution. EPCs may be responsible for endothelial regeneration, revascularization, secretion of thrombolytic enzymes and prevention of thrombus propagation and recurrence. By overexpressing a thrombolytic gene (tPA) in resident EPCs, innate capability of the EPCs to resolve thrombi may be enhanced and the process of thrombolysis may be accelerated.

Here it is proposed to further explore gene therapy for DVT using US-based gene delivery in a clinically relevant large-animal model. Specifically, the protocol of gene delivery is established, and its applicability for thrombolysis is tested.

Study design: In this aim the protocol of gene delivery to cells that reside within and adjacent to a femoral vein thrombus is developed. First, formation of a blood clot in the femoral vein of the minipig is induced and allowed to mature for one week. A reporter gene is then delivered to the vicinity of the thrombus. Efficiency of transfection and characterization of transfected cells may be performed 2, 5, and 10 days post-transfection. Experimental groups may include: 1) animals transfected with GFP plasmid (pGFP) mixed with MBs and US application; 2) animals treated with pGFP mixed with MBs, without US application; 3) animals treated with MBs and US application; and 4) non-treated animals, as detailed in Table 3. In addition, the effect of US-based gene delivery on the vein and adjacent tissues is evaluated. Primary outcome measures may include the percentage of cells transfected, analyzed by flow cytometry. Secondary outcomes may include surface markers analysis of transfected cells, using flow cytometry, and histology to evaluate tissue damage.

TABLE 3
Experimental Groups Aim 2

| Groups | Plasmid | MBs* | US | # of animals |
|---|---|---|---|---|
| Pilot study | — | Yes | Yes | 3 |
| pGFP + MBs + US | 1 mg | Yes | Yes | 18 |
| | | | | (n = 6/timepoint) |
| pGFP + MBs | 1 mg | Yes | — | 18 |
| | | | | (n = 6/timepoint) |
| MBs + US | — | Yes | Yes | 18 |
| | | | | (n = 6/timepoint) |
| Non treated | — | — | — | 18 |
| | | | | (n = 6/timepoint) |

*Amount to be determined in a pilot study

### Materials & Methods:

Reagents: A plasmid encoding for eGFP under a CMV promoter was purchased from GenScript and tested in vitro. Additional reagents to be used may include MBs (Definity, Lantheus) and commercial molecular biology reagents indicated below.

Animal model: All minipig studies may be conducted following Cedars-Sinai IACUC approval. To induce an aged thrombus formation in a minipig femoral vein, a model is adapted that was recently described by Stocker et al in In Vivo Porcine Aged Deep Vein Thrombosis Model for Testing Ultrasound-based Thrombolysis Techniques. Ultrasound Med Biol. 2021, which is incorporated by reference in its entirety.

Briefly, under general anesthesia two 8-Fr wedge balloon catheters (Arrow Al-07128, Teleflex, Morrisville, NC, USA) may be placed percutaneously via modified Seldinger technique in the femoral vein, with approximately 3-5 cm between them. First, a 21-gauge introducer needle (G43870, Cook Medical, Bloomington, IN, USA) may be used to gain access to the femoral vein. A 0.018-inch wire guide (G43870, Cook Medical, Bloomington, IN, USA) may be placed in the vein, and the introducer needle may be removed. The wire guide may be used to place a sterilized, reused dilator from a Terumo 10 cm 8 Fr sheath (RSS801, Terumo Medical Corporation, Elkton, MD, USA). The 0.018-inch wire guide may be exchanged with a 0.038-inch guide wire (GR3806, Terumo Interventional Systems, Somerset, NJ, USA) through the dilator. Finally, the wedge balloon catheter (Arrow Al-07128, Teleflex, Morrisville, NC, USA) may be placed over the 0.038-inch guide wire. This process may be completed with both balloon catheters. Next, 500 units of thrombin (RECOTHROM, ZymoGenetics, Seattle, WA, USA) may be injected through the distal catheter, and the clot may be allowed to form for 2-3 hours with the balloons inflated. Heparin at 200 units/kg may be given in a single bolus at the time of thrombin injection to anticoagulated the blood outside of the ballooned-off area. After clot formation, which may be verified by US imaging, the balloons may be deflated, and the distal catheter may be removed. The proximal catheter may be buried under the skin and serve as an anchor for the clot. Catheter insertion points may be closed with skin glue (Vetbond, 3M, Maplewood, MN, USA), and the animals may be recovered. Anti-coagulation may not be administered in the days following clot formation.

In vivo MB visualization and oscillation: Prior to the transfection study, three pilot animals in which a clot may be created may be used to determine the US parameters to be used with the probe developed above. One-week post-clot-formation, under general anesthesia, thrombus location and dimension may be verified using US. B-mode and doppler imaging may be used to visualize the clot and estimate the percentage of vein occlusion, as described by Stocker et al. Then MBs may be injected via catheter distally to the thrombus location, at different concentrations. The US parameters required to visualize and oscillate MBs at the target may then be determined.

In vivo transfection: In minipigs with one or more established clots in the femoral vein, 1 mg of GFP plasmid mixed with MBs (Definity) may be injected through a catheter. Immediately after injection, therapeutic US may be applied transcutaneously to oscillate the MBs until they are no longer visible.

In-life monitoring: Animals may be monitored for any signs of adverse events, especially respiratory difficulties, and pain.

Postmortem analyses: Animals may be euthanized 2-, 5- and 10-days post-transfection (i.e., 9-, 14- and 17-days post-clot-formation). Segments of the femoral vein containing clot and transfection region may be collected and subjected to the analyses detailed below. Contralateral veins may serve as internal controls, and femoral veins from untreated animals may be used as negative controls.

Ex vivo fluorescence: Vein explants may be subjected to fluorescence imaging (IVIS, PerkinElmer).

Flow cytometry: Following ex vivo fluorescence imaging, cells may be isolated from vein segments and thrombi. Briefly, tissues may be washed with phosphate buffered saline (PBS), digested using 0.1% collagenase (type 1A, Sigma-Aldrich) for one hour, filtrated using a 70-mm cell strainer, and centrifuged at 2000 rpm for 7 minutes. Freshly isolated cells may be analyzed using flow cytometry for expression of EPC, smooth muscle, endothelial, fibroblast, and inflammatory cell surface markers (Abcam), according to Table 4.

TABLE 4
Cell Surface Markers

| | | Absence of |
|---|---|---|
| Cell type | Presence of markers | markers* |
| EPC | CD34, VEGFR2, CD133 | — |
| EC | CD31, CD146, CD105, von Willebrand | CD14, CD145, |
| | Factor (vWF) | CD146 |
| Fibroblast | Factor VIII, cytokeratin 19, α-actin | vWEF |
| Smooth muscle | α-actin | CD31 |

Histology: In each animal, part of the vein containing the clot may be removed and fixed in 10% phosphate buffered formalin before being embedded in paraffin. Tissue blocks may then be sectioned and stained with hematoxylin and eosin (H&E) and Martius Scarlet Blue MSB). Clots may be categorized as phase I-III according to characteristics described by Fineschi et al in Histological age determination of venous thrombosis: a neglected forensic task in fatal pulmonary thrombo-embolism. Forensic Sci Int. 2009;186(1-3):22-8, which is incorporated by reference herein in its entirety.

Immunohistochemistry: Part of the paraffin-embedded sections may be used for detection of GFP using anti-GFP antibody at 1/400 dilution (Alexa Fluor 555 conjugated, rabbit polyclonal IgG (indirect detection, cat#A-31851, ThermoFisher), as described by Sper et al.

Statistical Considerations: Data may be tested across groups with factorial ANOVA. With 6 animals per group per timepoint, there is at least 80% power to detect the expected difference of at least 12% across the interaction of group and time factors in GFP+ transfection rates assuming a SD=22.5%.

### Example 5: Induction of in Vivo Thrombolysis Using US-Mediated Gene Delivery

A localized secretion of active rtPA over the course of several days may induce efficient thrombus resolution in a physiological manner, reducing the unwarranted risk of hemorrhage reported for the systemic use of Alteplase. In comparison to US-assisted, catheter-directed thrombolysis (without gene therapy), which requires endovascular treatment of 15-45 hours, the method of treatment described herein (that is, US-assisted microbubble or nanobubbles-based gene therapy) could be accomplished within 30 minutes. In addition, suggested transcutaneous methods of thrombolysis (e.g., microtripsy and histotripsy) might require 16minutes of US application per 1 cm clot, as shown in a pig model, which could enhance the risk of tissue and vessel damage. In one example, the gene for tPA may be overexpressed, as this thrombolytic factor has been shown to promote EPC recruitment via the upregulation of SDF-1a. Further, the inventors herein have recognized that by recruitment of additional EPCs to the thrombus site in addition to the local effect of rtPA expression, a synergistic effect of thrombolysis would be achieved. Accordingly, in some examples, as discussed above targeting microbubbles or nanobubbles that include targeting antibodies to recognize target cells (e.g., EPCs) may be used for targeted gene therapy to transfect target cells and induce expression or overexpression of the therapeutic agent (e.g., tPA or rtPA) in the target cells to further improve the efficacy of thrombolysis.

Study design: In this aim the transfection protocol and the gene delivery protocol discussed above may be used to induce efficient rtPA gene transfection in a minipig DVT model. First, DVT may be induced in the femoral veins of minipigs. One week later, tPA gene delivery may be performed and its effect on thrombus resolution may be examined. Primary outcomes may include thrombus size and blood flow to be measured by in vivo US imaging. Secondary outcomes may include the safety aspects of the treatment specifically—blood levels of rtPA, local damage to the treated vein and the presence of emboli in the lung tissue. Experimental groups may include: 1) animals transfected with rtPA plasmid mixed with MBs and US application; 2) animals treated with rtPA plasmid mixed with MBs, without US application; 3) animals treated with MBs and US application; 4) animals treated with Alteplase (standard of care); and 5) non-treated animals, as detailed in Table 5.

TABLE 5
Experimental Groups Aim 3

| Groups | Plasmid | MBs* | US | # of animals |
|---|---|---|---|---|
| ptPA + MBs + US | 1 mg | Yes | Yes | 8 |
| ptPA + MBs | 1 mg | Yes | — | 8 |
| MBs + US | — | Yes | Yes | 8 |
| Alteplase | — | — | — | 8 |
| Non treated | — | — | — | 8 |

*Amount to be determined in a pilot study

Materials & methods:

Reagents: A plasmid encoding for human tPA under a CMV promoter was purchased from Genscript and tested in vitro (FIG. 7). Additional reagents to be used may include MBs (Definity, Lantheus) and commercial molecular biology reagents indicated below.

Animal model: A minipig DVT model may be used as described above.

In vivo transfection: A transfection protocol established above may be used, and the US transducer developed above may be applied.

In-life monitoring: Blood flow analysis: Thrombus size and blood flow through the treated femoral vein may be evaluated using Doppler US and phlebography pre-treatment and on Days 4, 7 and 14 post-transfections.

Doppler imaging may be performed in pig models of DVT: using a linear imaging probe linked to a Verasonics US scanner (Verasonics, Vantage 256), Doppler longitudinal and transverse sections may be generated, as well as pulsed Doppler, to measure blood flow velocity measurement. The permeability of the vein after recanalization may be shown by the reappearance of permanent flow with color Doppler and partial vein compression. Phlebography may be performed after injection of a contrast agent (Iomeron®, IOMEPROL 816 mg/ml, BraccoC) through the catheter in the distal femoral vein. Before treatment, the occlusive nature of the thrombosis may be demonstrated by stagnation of the contrast agent in the distal femoral vein with no contrast in the iliac vein. After treatment, extent of recanalization may be shown by passage of the contrast agent from the distal femoral vein to the iliac vein through the recanalized channel, and integrity of the venous wall by the absence of contrast-agent extravasation.

Serum tPA levels: In order to analyze human rtPA levels adjacent to the thrombus and in circulation, blood samples may be taken from the femoral vein (distal to thrombus using a catheter) and ear vein on Days 4, 7 and 14 post-transfections, while the animals are anesthetized for US imaging and phlebography. Human rtPA levels may be measured using a sandwich (ThermoFisher). The levels of porcine tPA levels may be assessed using a designated (Biomatik, Cat#EKU06668).

Postmortem analyses: Animals may be euthanized 14 days post-transfection.

Clot size: Following euthanasia, clots from the femoral veins may be excised, measured, and weighed to evaluate the effect of treatment.

Histopathology: To determine the effect of treatment on the femoral vein and surrounding tissues, histological analysis may be done as described above. In addition, immediately after euthanasia, the lungs may be perfused with paraformaldehyde through the main bronchi. Macroscopic analyses of the pulmonary arteries and lung parenchyma may be conducted to search for presence of bulky emboli and pulmonary infarction foci. Microscopic analysis may be done on tissue samples from each lobe to assess the presence of microscopic thrombus debris in the pulmonary arteries and pulmonary infarction. To better examine endothelial cell damage, the vein sections may be stained with an anti-von Mayebrand antibody (Abcam, ab6994).

Statistical Considerations: Data collected in vivo over time may be tested with RM-ANOVA. Post-mortem data with ANOVA. With a sample size of 8 per group there may be 88% power to detect the expected difference of at least 12 mm² (69) in the decrease of thrombus size over time between groups at the 0.05 level assuming SD=12 mm², a between group effect of at least 16 mm² (82% power), a within subject effect of 5 mm² (88% power), and autocorrelations of 0.65.

### Computer & Hardware Implementation of Disclosure

It should initially be understood that the disclosure herein may be implemented with any type of hardware and/or software. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

In some implementations, the computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine- generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### Conclusion

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A method for theragnosis, treating, ameliorating, or preventing a vascular condition in a subject, the method comprising:
administering a composition to the subject at or near a region of interest including one or more blood vessels, the composition comprising an ultrasound contrast agent and a gene therapy vector including at least a gene for a therapeutic agent for treatment, amelioration, or prevention of the vascular condition and a sequence that causes the therapeutic agent to be expressed in the subject; and
imaging, via an ultrasound probe, the microbubbles in the region of interest using a contrast pulse sequence of ultrasound plane waves; and
applying, via the ultrasound probe, a therapeutic ultrasound emission for transfection of the gene therapy vector into a plurality of vascular cells of the one or more blood vessels;
wherein the ultrasound probe is configured to image the one or more blood vessels, one or more occlusions associated with the one or more blood vessels, and/or microbubbles in the one or more blood vessels and configured to provide transcutaneous ultrasound therapy to the one or more blood vessels at a depth below the skin surface.

2. The method of claim 1, further comprising, imaging, via the ultrasound probe, the one or more blood vessels and/or the one or more occlusions before administering the composition to the subject.

3. The method of claim 1, wherein the therapeutic agent is a selected from the group consisting of tissue plasminogen activator, recombinant tissue plasminogen factor, urokinase, plasminogen, and streptokinase.

4. The method of claim 1, wherein the one or more occlusions comprises one or more thrombus and/or one or more embolus.

5. The method of claim 1, wherein the ultrasound contrast agent comprises microbubbles or nanobubbles.

6. The method of claim 5,
wherein the microbubbles are synthetic or biological-shelled microbubbles, or
wherein the microbubbles are in a size range between 0.5-15 micrometers (μm), or
wherein a therapeutic concentration of the microbubbles is in a range between 1×10⁶/1ml 1×10¹⁰/1 ml, or
wherein the nanobubbles are in a size range between 200-800 nanometers (nm), or
wherein the microbubbles or nanobubbles are modified with targeting antibodies to a target cell, or
wherein the microbubbles or nanobubbles are conjugated with the gene therapy vector.

7. (canceled)

8. (canceled)

9. (canceled)

10. (canceled)

11. The method of claim 1,
wherein the gene therapy vector is a plasmid DNA, or
wherein the gene therapy vector includes a microRNA (miRNA) expression sequence or wherein the gene therapy vector is a miRNA expression vector for reducing therapeutic gene expression in non-targeted cells, or
wherein the gene therapy vector is a long non-coding RNA expression vector.

12. (canceled)

13. (canceled)

14. (canceled)

15. The method of claim 6, wherein the target cell is an endothelial cell, endothelial progenitor cell, or muscle cell.

16. (canceled)

17. (canceled)

18. A method for treating, ameliorating, or preventing one or more vascular conditions in a subject, the method comprising:
administering a composition to the subject at or near a region of interest including one or more blood vessels, the composition comprising an ultrasound contrast agent and a nucleic acid sequence for expression of a therapeutic agent into one or more target cells for treatment, amelioration, or prevention of the one or more vascular conditions; and
applying, via an ultrasound probe, a therapeutic ultrasound emission for transfection of the nucleic acid sequence into one or more target cells of the one or more blood vessels.

19. The method of claim 18, wherein the nucleic acid sequence is a plasmid DNA including a gene encoding the therapeutic agent or a synthetic mRNA that when transfected into the cell is translated into the therapeutic agent.

20. The method of claim 18, wherein the vascular condition is selected from the group consisting of DVT, PE, venous thromboembolism, post-thrombotic syndrome, embolic strokes, embolic heart attack, and combinations thereof.

21. The method of claim 18, wherein the therapeutic agent is a selected from the group consisting of tissue plasminogen activator, recombinant tissue plasminogen factor, urokinase, plasminogen, and streptokinase.

22. The method of claim 18, wherein the ultrasound contrast agent comprises microbubbles or nanobubbles or a combination of microbubbles and nanobubbles.

23. The method of claim 22,
wherein the nanobubbles are in a size range between 200-800 nm, or
wherein the microbubbles are in a size range between 0.5-15 μm, or
wherein the microbubbles or nanobubbles are modified with targeting antibodies to the one or more target cells, or
wherein the microbubbles or nanobubbles are conjugated with the nucleic acid sequence, or
wherein the modified microbubbles or nanobubbles are conjugated with the gene therapy vector.

24. (canceled)

25. The method of claim 18, wherein the nucleic acid sequence is a plasmid DNA, or wherein the nucleic acid sequence comprises a miRNA expression sequence, or a long non-coding RNA expression sequence.

26. (canceled)

27. (canceled)

28. The method of claim 18, wherein the one or more target cells is selected from the group consisting of an endothelial cell, an endothelial progenitor cell, a muscle cell, or a combination thereof.

29. (canceled)

30. (canceled)

31. A composition for treatment of one or more vascular conditions in a subject comprises: a microbubble or a nanobubble; and a vector including a gene encoding a therapeutic agent for the treatment of the one or more vascular conditions.

32. The composition of claim 31,
wherein the vascular condition is selected from the group consisting of DVT, PE, venous thromboembolism, post-thrombotic syndrome, embolic strokes, embolic heart attack, and combinations thereof, or
wherein the one or more target cells is selected from the group consisting of an endothelial cell, an endothelial progenitor cell, a smooth muscle cell, or a combination thereof.

33. The composition of claim 31, wherein the therapeutic agent is a selected from the group consisting of tissue plasminogen activator, recombinant tissue plasminogen factor, urokinase, plasminogen, and streptokinase.

34. The composition of claim 31, wherein the microbubbles or nanobubbles are modified with targeting antibodies to the one or more target cells.

35. (canceled)

36. The composition of claim 31,
wherein the vector is plasmid DNA or mRNA, or
wherein the vector comprises a miRNA expression sequence, or a long non-coding RNA expression sequence.

37. (canceled)

38. A therapeutic composition for ultrasound-assisted gene therapy, the therapeutic composition comprising an ultrasound contrast agent comprising microbubbles or nanobubbles, and a gene therapy vector including a gene encoding a therapeutic agent for dissolution of thrombosis and/or embolus in a blood vessel.

39. The therapeutic composition of claim 38, wherein the therapeutic agent is a selected from the group consisting of tissue plasminogen activator, recombinant tissue plasminogen factor, urokinase, plasminogen, and streptokinase.

40. The therapeutic composition of claim 38, wherein the microbubbles or nanobubbles are modified with targeting antibodies to the one or more target cells.

41. The therapeutic composition of claim 38, wherein the one or more target cells is selected from the group consisting of an endothelial cell, an endothelial progenitor cell, a muscle cell, or a combination thereof.

42. The therapeutic composition of claim 38, wherein the vector is plasmid DNA or mRNA, wherein the vector comprises a miRNA expression sequence, or a long non-coding RNA expression sequence.

43. (canceled)

44. A method for theragnosis, treating, ameliorating, or preventing of one or more vascular conditions in a subject, the method comprising:
providing a composition of claim 31 at or near a region of interest for the subject;
performing contrast imaging of the region of interest, via an ultrasound probe, using an imaging ultrasound sequence; and
transmitting an ultrasound sequence, via the ultrasound probe, for transfection of the vector in to a plurality of target cells at or near the region of interest.

45. A kit comprising:
an ultrasound contrast agent comprising microbubbles, nanobubbles, or both;
a vector comprising a nucleic acid sequence encoding a therapeutic agent for treating, ameliorating, or preventing a vascular disease; and
instructions for use.

46. (canceled)

47. The kit of claim 45,
wherein the vector further comprises a sequence that causes the therapeutic agent to be expressed in the subject,
wherein each of the microbubbles have a shell, the shell selected from the group consisting of proteins, phospholipids, biodegradable polymeric material, surfactant, and multi-layer polyelectrolytes, or
wherein the therapeutic agent is selected from the group consisting of tissue plasminogen activator, recombinant tissue plasminogen factor, urokinase, plasminogen, streptokinase.

48. (canceled)

49. (canceled)

50. An ultrasound system comprising:
an ultrasound probe;
a non-transitory memory having instructions stored therein; and
a controller configured to execute the instructions to:
during a first imaging mode, transmit, via the ultrasound probe, a first plane wave sequence at a first transmission frequency to image one or more blood vessels and/or associated occlusions in a volumetric region of interest; and
during a second imaging mode, transmit, via the probe, a second plane wave sequence at a second transmission frequency to image a plurality of microbubbles within the one or more blood vessels in the volumetric region of interest.

51. An ultrasound system for diagnosis and treatment of a vascular condition of one or more blood vessels, the ultrasound system comprising:
an ultrasound probe that includes an array of transducer elements;
a non-transitory memory having instructions stored therein; and
a controller configured to execute the instructions to:
during a first imaging mode, acquire, via the ultrasound probe, a first set of ultrasound image data of one or more blood vessels and/or an associated occlusion in a volume of interest; and
during a second imaging mode, acquire, via the ultrasound probe, a second set of ultrasound image data of a plurality of microbubbles in the one or more blood vessels and/or an associated occlusion in a volume of interest.

52. The ultrasound system of claim 51,
wherein the controller is further configured to execute the instructions to:
during a third therapeutic mode, provide, via the ultrasound probe, a set of therapeutic ultrasound emissions to insonate the volume of interest for providing microbubble-assisted gene therapy, or
wherein the controller is further configured to execute the instructions to:
process the first set of ultrasound image data, in real-time or near real-time, to generate a volumetric rendering the one or more blood vessels and/or the associated occlusion; and
process the second set of ultrasound image data, in real-time or near real-time, to generate a second volumetric rendering the plurality of microbubbles in the one or more blood vessels and/or the associated occlusion, or
wherein the first set of ultrasound image data is generated using a first transmission sequence for B-mode imaging at a first transmission frequency; and wherein the second set of ultrasound image data is generated using a second transmission sequence for contrast pulse sequence (CPS) imaging at a second transmission frequency, the second transmission frequency less than the first transmission frequency.

53. (canceled)

54. (canceled)
